SEQUENCE LISTING (12 sequences)

SEQ ID NO: 1 - protein, 27 aa, synthetic construct
KTKATVLIKNKQKSKNALKQKIVLLSK

SEQ ID NO: 2 - protein, 27 aa, synthetic construct
LKTKIKIIVKTKSSAKLRSKLVNSHKI

SEQ ID NO: 3 - protein, 27 aa, synthetic construct
TQLRNKYTFLARARNALAVRTKQNIKS

SEQ ID NO: 4 - protein, 27 aa, synthetic construct
TNLRNKYTFLARARANLAVRNKQNIKS

SEQ ID NO: 5 - protein, 27 aa, synthetic construct
KTKATVKIKNKQKSVNALKQKIVLLSK

SEQ ID NO: 6 - protein, 27 aa, synthetic construct
LKQKIKHVVKLKVVVKLRSQLVKRKQN

SEQ ID NO: 7 - protein, 27 aa, synthetic construct
KKKKKLQLQLNLIKKKVQVSVVVVRRH

SEQ ID NO: 8 - protein, 26 aa, synthetic construct
TKSKIKIVVKSKAKKLRLALVKRIKI

SEQ ID NO: 9 - protein, 27 aa, synthetic construct
TKTKIKIIVKLKSKAKLRIKLVKRHKS

SEQ ID NO: 10 - protein, 28 aa, synthetic construct
ASNLTKAAKSLKVRVIKKTKQKQVLKVL

SEQ ID NO: 11 - protein, 23 aa, synthetic construct
TKAKIKHSVKLKAKLRIKLVKRI

SEQ ID NO: 12 - protein, 27 aa, synthetic construct
LKLKSSHSIKLKVKSKQRSALVSRQKA

(19) United States
(12) Patent Application Publication
(10) Pub. No.: US 2025/0026792 A1
(43) Pub. Date: Jan. 23, 2025

(54) SELF-ASSEMBLING PEPTIDES WITH HYALURONIC ACID BINDING DOMAINS AND METHODS OF USE THEREOF

(71) Applicant: Northwestern University, Evanston, IL (US)

(72) Inventors: Kyle Michael Koss, Evanston, IL (US); Terrance J. Sereda, Lamont, CA (US); Jason Albert Wertheim, Evanston, IL (US)

(21) Appl. No.: 18/852,565

(22) PCT Filed: Mar. 31, 2023
(86) PCT No.: PCT/US2023/065192

§ 371 Date: Sep. 30, 2024

Related U.S. Application Data
(60) Provisional application No. 63/326,338, filed on Apr. 1, 2022.

(51) Int. Cl. C07K 14/00

(52) U.S. Cl. CPC C07K 14/001

(57) ABSTRACT

Provided herein are self-assembling peptides comprising hyaluronic acid binding domains, nanofibers and systems comprising the same, and methods of use thereof.

### STATEMENT REGARDING RELATED APPLICATIONS

This application claims priority to U.S. Provisional Patent Application No. 63/326,338, filed Apr. 1, 2022, the entire contents of which are incorporated herein by reference for all purposes.

### SEQUENCE LISTING

The text of the computer readable sequence listing filed herewith, titled “NWEST-40734-601_SQL”, created Mar. 29, 2022, having a file size of 11,685 bytes, is hereby incorporated by reference in its entirety.

### FIELD OF INVENTION

Provided herein are self-assembling peptides comprising hyaluronic acid binding domains, nanofibers and systems comprising the same, and methods of use thereof.

### BACKGROUND

Self-assembling peptides have been used in various research efforts with applications to molecular biology and medicine. In some instances, self-assembling peptides may be used in combination with therapeutic agents to treat a condition or disorder in a subject. However, efficacy and viability of the therapeutic agent in vivo can be limited. Accordingly, novel self-assembling peptides are needed.

### SUMMARY

In some aspects, provided herein are self-assembling peptides. In some embodiments, provided herein is a self-assembling peptide comprising a plurality of BX₇B domains, wherein B is a basic amino acid and X is any amino acid except an acidic amino acid. In some embodiments, the self-assembling peptide binds to one or more extracellular matrix components. For example, in some embodiments the self-assembling peptide binds to collagen, elastin, and/or hyaluronic acid.

In some embodiments, the self-assembling peptide comprises 26-28 amino acids.

In some embodiments, the self-assembling peptide comprises three, four or five BX₇B domains. In some embodiments, the self-assembling peptide comprises at least two contiguous BX₇B domains and at least two non-contiguous BX₇B domains; a first set of at least two contiguous BX₇B domains and a second set of at least two contiguous BX₇B domains, each set is non-contiguous with the other set; or at least three BX₇B domains, wherein two of the at least three BX₇B domains are contiguous with each other and the third BX₇B domain is non-contiguous with the other two BX₇B domains.

In some embodiments, B is a basic amino acid selected from histidine, arginine, or lysine. In some embodiments, X is an amino acid selected from histidine, lysine, arginine, serine, threonine, asparagine, glutamine, alanine, valine, leucine, isoleucine, methionine, phenylalanine, tyrosine, tryptophan, proline, glycine, and cysteine. In some embodiments, the self-assembling peptide has a propensity to form β-sheet secondary structure.

In some embodiments, the self-assembling peptide comprises a sequence selected from SEQ ID NO: 1, SEQ ID NO: 2, SEQ ID NO: 3, SEQ ID NO: 4, and SEQ ID NO: 5.

In some aspects, provided herein is a nanofiber comprising a plurality of self-assembling peptides. In some embodiments, each of the plurality of self-assembling peptides comprises a plurality of BX₇B domains, wherein B is a basic amino acid and X is any amino acid except an acidic amino acid, wherein the self-assembling peptide binds to hyaluronic acid. In some embodiments, each of the plurality of self-assembling peptides comprises three, four or five BX₇B domains. In some embodiments, each of the plurality of self-assembling peptides comprises at least two contiguous BX₇B domains and at least two non-contiguous BX₇B domains; a first set of at least two contiguous BX₇B domains and a second set of at least two contiguous BX₇B domains, each set is non-contiguous with the other set; or at least three BX₇B domains, wherein two of the at least three BX₇B domains are contiguous with each other and the third BX₇B domain is non-contiguous with the other two BX₇B domains.

In some embodiments, B is a basic amino acid selected from histidine, arginine, or lysine. In some embodiments, X is an amino acid selected from histidine, lysine, arginine, serine, threonine, asparagine, glutamine, alanine, valine, leucine, isoleucine, methionine, phenylalanine, tyrosine, tryptophan, proline, glycine, and cysteine. In some embodiments, each self-assembling peptide in the nanofiber has a propensity to form 3-sheet secondary structure. In some embodiments, each self-assembling peptide in the nanofiber comprises a sequence independently selected from SEQ ID NO: 1, SEQ ID NO: 2, SEQ ID NO: 3, SEQ ID NO: 4, and SEQ ID NO: 5.

In some aspects, provided herein is a system comprising a self-assembling peptide described herein bound to hyaluronic acid. In some embodiments, the system comprises a hydrogel.

In some aspects, provided herein are materials coated with a nanofiber or a system described herein. For example, in some embodiments the material is a biomedical device, such as a neural implantable device.

In some embodiments, the self-assembling peptides, nanofibers, and systems described herein find use in methods of treating inflammatory conditions, cancer, and promoting wound healing in a subject.

Additional embodiments will be apparent to persons skilled in the relevant art based on the teachings contained herein.

### BRIEF DESCRIPTION OF THE DRAWINGS

FIGS. 1A-1B shows an overview of an exemplary process for peptide design and evaluation of peptide binding and biophysics. (FIG. 1A) An example illustration of mapping single B(X₇)B hyaluronic acid binding site in mPEP35, showing (i) N to C termini (blue to red) ribbon model, (ii) side and overview stick model with lysine 4 (K4), valine 8 (V8), and lysine 12 (K12) spatial locations, and (iii) helical net with drawn line (B(X₇)B domain) between these residues. The domain is drawn by connecting the two positive charges, i.e., B1 (residue #4) and B2 (residue #12) of a B(X7)B domain, where I5 to L11 are the residues in the C7 region of the B(X7)B domain. (FIG. 1B) Secondary structure shift from 3₁₀ helical, to beta sheet (intra/intermolecular), and eventually self-assembled nanofiber with increase peptide concentration.

FIGS. 2A-2L. Example helical net plots and 3D models of (FIG. 2A) mPEP35, (FIG. 2B) 1-scrm, (FIG. 2C) 17x-3, (FIG. 2D) 6f-2, (FIG. 2E) 8h-2, (FIG. 2F) 2nd de novo, (FIG. 2G) 10K, (FIG. 2H) 4, (FIG. 2I) 6b, (FIG. 2J) 7c, (FIG. 2K) BHP3, and (FIG. 2L) BHP4. In the helical net plots, blue spots, yellow, and white spots represent amino acids with positive, non-polar, and polar residues, respectively. Blue lines trace the predicted B(X₇)B domains. Amino acids are ordered, from N to C terminus, diagonally downwards from right to left and are numbered accordingly. In the model, N to C termini are presented blue to red in the color spectra. Modelling was done on the PEP-FOLD server.¹⁹

FIG. 3 shows concentration-dependent peptide binding to hyaluronic acid characterized by fluorescence intensity (Ex 490 nm, Em525 nm), through a biotin-streptavidin interaction. Peptides were incubated 24 hours at 37° C. Coatings (10 μg/cm²) were previously dried overnight in milli-Q water. Overall significance was assessed using analysis of variance, with Tukey's multiple comparison post-hoc analysis between 1-scrm and experimental groups where n=4.

FIG. 4 shows extra-cellular dependent peptide binding characterized by fluorescence intensity (Ex 490 nm, Em525 nm), through a biotin-streptavidin interaction. Peptides were incubated 24 hours at 37° C. on collagens I-IV, elastin, hyaluronic acid, and Geltrex. Coatings (10 pig/cm²) were previously dried overnight in milli-Q water. Overall significance was assessed using analysis of variance, with Tukey's multiple comparison post-hoc analysis between ECM groups where n=4.

FIGS. 5A-5L shows circular dichroism (CD) spectral profiles of (FIG. 5A) mPEP35, (FIG. 5B) 1-scrm, (FIG. 5C) 17x-3, (FIG. 5D) 6f-2, (FIG. 5E) 8h-2, (FIG. 5F) 2^{nd} de novo, (FIG. 5G) 10K, (FIG. 5H) 4, (FIG. 5I) 6b, (FIG. 5J) 7c, (FIG. 5K) BHP3, and (FIG. 5L) BHP4 in 20 mM sodium cacodylate (SC), pH 7.4, (i) without and (ii) with trifluoroethanol (TFE). Data is presented in ellipticity ([θ]_{M}×10⁻³) as a function of spectral wavelength (λ). Peptides were sonicated for 30 minutes at concentrations 1.25, 2.5, and 5 mg/ml, and incubated at 37° C. for 24 hours before peptides were pipetted into a 0.5 mm type 20 demountable 0-shaped quartz cuvette.

FIGS. 6A-6L shows transmission electron microscopy of (FIG. 6A) mPEP35, (FIG. 6B) 1-scrm, (FIG. 6C) 17x-3, (FIG. 6D) 6f-2, (FIG. 6E) 8h-2, (FIG. 6F) 2^{nd} denovo, (FIG. 6G) 10K, (FIG. 6H) 4, (FIG. 6I) 6b, (FIG. 6J) 7c, (FIG. 6K) BHP3, and (FIG. 6L) BHP4 in (i) phosphate buffered saline (PBS), and (ii) 20 mM sodium cacodylate (SC), both pH 7.4. All samples were stained with 4% uranyl acetate and imaged at 0.5% w/v in TNC buffer after 30 minutes of sonication and 24 hrs of incubation at 37° C.

FIGS. 7A-7C show Nuclear magnetic resonant (NMR) proton spectra of representative (i) hyaluronic acid peaks and (ii) biotinyl peptide peaks and for (FIG. 7A) mPEP35, (FIG. 7B) 1-scrm, and (FIG. 7C) 17x-3 in 25 mM sodium acetate buffer at pH 5.2. Samples represent supernatant (soluble component) of mixtures with 5 mg/mL HA in variable peptide mixture of 1.25, 2.5. and 5 mg/mL.

FIGS. 8A-8C shows Fourier Transform Infrared (FTIR) of representative spectral profiles of (FIG. 8A) mPEP35, (FIG. 8B) 1-scrm, and (FIG. 8C) 17x-3 in 20 mM sodium cacodylate (SC), pH 7.4 with 20% trifluoroethanol (TFE). Data is presented in ellipticity ([θ]_{M}×10⁻³) as a function of spectral wavelength (λ). Peptides were sonicated for 30 minutes at concentrations 1.25, 2.5, and 5 mg/mL, and incubated at 37° C. for 24 hours before peptides were onto the detection crystal.

FIGS. 9A-9C show multimolecular in silico simulation of peptide 17x-3. Peptides were initiated as alpha helical structures with the intent of visualizing realignment to beta sheet/self-assembly reorganization. (FIG. 9A) Overall representation, (FIG. 9B) an emphasis on isolated bridges (pre-beta sheets), and (FIG. 9C) and uncoiled alpha helixes after 50 ns. Pink and purple note a mixture of alpha and 3₁₀ helices, respectively FIG. 10 shows nuclear magnetic resonance (NMR) spectra of chemical shift, nuclear Ovehauser effect (NOE), and J-coupling of peptide 17x-3 at 5 mg/ml in sodium acetate buffer without and with 20% trifluoroethanol. Spectra and assignments include ¹⁵N heteronuclear single quantum coherence (¹⁵N-HSQC), ¹³C-HSQC, total correlation spectroscopy (TOCSY)/NOESY H_{N}-H_{α} regions, and NOESY H_{N} regions. Included are examples of which protons in carbonyls and NHs were selected per spectra.

FIG. 11 shows TALOS-N chemical shift analysis and prediction of 17x-3 based on assignments and shifts from 2D spectras including ¹⁵N-HSQC, ¹³C-HSQC, TOCSY/NOESY H_{N}-H_{α} regions, and NOESY H_{N}. NMR spectras were collected of peptide 17x-3, 5 mg/ml, in sodium acetate buffer (pH 5.4), without and with 20% trifluoroethanol.

### DETAILED DESCRIPTION

Described herein is a novel class of self-assembling peptides designed with B(X₇)B hyaluronic acid binding domains. The peptides exhibit robust hyaluronic acid (HA) binding behavior.

Binding to extra-cellular domains (ECM) allows for a variety of novel applications involving tissues exposed in injury, cancer, or implantable devices. Ten different peptides were assessed. Simple molecular modelling was used to evaluate secondary structures, concentration and extra-cellular dependent binding assay were performed, concentration mediated secondary structures were assessed using circular dichroism (CD), and higher order nanostructures were visualized using transmission electron microscopy (TEM). All peptides formed the initial apparent 3₁₀/helical shapes, however peptides termed 17x-3, 4, BHP3 and BHP4 were found to be HA specific and robust binders, especially at higher concentrations. It was noted from CD-derived ellipticity ratios, that these peptides shifted from the initially apparent 3₁₀/alpha-helical shapes at lower concentrations (1.25 mg/ml) to beta-sheets at higher concentrations (5 mg/ml). Furthermore, these binders also formed nanofibers noted common self-assembling structures typically cued by beta-sheet formation. As such, provided herein are HA binding peptides that outperform mPEP35 (positive commercial standard) 3-4 times at higher concentrations, which was enhanced by self-assembly.

### 1. Definitions

Although any methods and materials similar or equivalent to those described herein can be used in the practice or testing of embodiments described herein, some preferred methods, compositions, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a peptide amphiphile” is a reference to one or more peptide amphiphiles and equivalents thereof known to those skilled in the art, and so forth.

As used herein, the terms “comprise”, “include”, and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

The term “amino acid” refers to natural amino acids, unnatural amino acids, and amino acid analogs, all in their D and L stereoisomers, unless otherwise indicated, if their structures allow such stereoisomeric forms.

Natural amino acids include alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V).

Unnatural amino acids include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethylglycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), N-alkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“OctG”), ornithine (“Orn”), pentylglycine (“pG” or “PGly”), pipecolic acid, thioproline (“ThioP” or “tPro”), homoLysine (“hLys”), homoArginine (“hArg”), and homoserine (“Hse”).

The term “amino acid analog” refers to a natural or unnatural amino acid where one or more of the C-terminal carboxy group, the N-terminal amino group and side-chain bioactive group has been chemically blocked, reversibly or irreversibly, or otherwise modified to another bioactive group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide and S-(carboxymethyl)-cysteine sulfone.

As used herein, the term “artificial” refers to compositions and systems that are designed or prepared by man and are not naturally occurring. For example, an artificial peptide, peptoid, or nucleic acid is one comprising a non-natural sequence (e.g., a peptide without 100% identity with a naturally-occurring protein or a fragment thereof).

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another:
1) Alanine (A) and Glycine (G);
2) Aspartic acid (D) and Glutamic acid (E);
3) Asparagine (N) and Glutamine (Q);
4) Arginine (R) and Lysine (K);
5) Isoleucine (I), Leucine (L), Methionine (M), and Valine (V);
6) Phenylalanine (F), Tyrosine (Y), and Tryptophan (W);
7) Serine (S) and Threonine (T); and
8) Cysteine (C) and Methionine (M).

Naturally occurring residues may be divided into classes based on common side chain properties, for example: polar positive (or basic) (histidine (H), lysine (K), and arginine (R)); polar negative (or acidic) (aspartic acid (D), glutamic acid (E)); polar neutral (serine (S), threonine (T), asparagine (N), glutamine (Q)); non-polar aliphatic (alanine (A), valine (V), leucine (L), isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs.

Non-conservative substitutions may involve the exchange of a member of one class for a member from another class or for a non-naturally occurring amino acid residue that does not share chemical properties with the substituted residue.

As used herein, the term “peptide” refers an oligomer to short polymer of amino acids linked together by peptide bonds. In contrast to other amino acid polymers (e.g., proteins, polypeptides, etc.), peptides are of about 50 amino acids or less in length. A peptide may comprise natural amino acids, non-natural amino acids, amino acid analogs, and/or modified amino acids. A peptide may be a subsequence of naturally occurring protein, a non-natural (artificial) sequence (in some embodiments, with possible acetylation/amidation), or a peptide analogue. A peptide may comprise one or more modifications, such as an N-terminal acetylation and/or a C-terminal amidation.

As used herein, the term “sequence identity” refers to the degree of which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ by amino acid substitutions (including conservative, semi-conservative, and non-conservative substitutions). The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

Any polypeptides described herein as having a particular percent sequence identity or similarity (e.g., at least 70%) with a reference sequence ID number, may also be expressed as having a maximum number of substitutions (or terminal deletions) with respect to that reference sequence. For example, a sequence having at least Y % sequence identity (e.g., 90%) with SEQ ID NO:Z (e.g., 100 amino acids) may have up to X substitutions (e.g., 10) relative to SEQ ID NO:Z, and may therefore also be expressed as “having X (e.g., 10) or fewer substitutions relative to SEQ ID NO:Z.”

As used herein, the term “biocompatible” refers to materials and agents that are not toxic to cells or organisms. In some embodiments, a substance is considered to be “biocompatible” if its addition to cells in vitro results in less than or equal to approximately 10% cell death, usually less than 5%, more usually less than 1%.

As used herein, the term “nanofiber” refers to an elongated or threadlike filament (e.g., having a significantly greater length dimension that width or diameter) with a diameter typically less than 100 nanometers. In some embodiments, the “nanofiber” is a ribbon-like filament, such as a twisted ribbon-like thread.

As used herein, “biodegradable” as used to describe the polymers, hydrogels, and/or wound dressings herein refers to compositions degraded or otherwise “broken down” under exposure to physiological conditions. In some embodiments, a biodegradable substance is a broken down by cellular machinery, enzymatic degradation, chemical processes, hydrolysis, etc. In some embodiments, a wound dressing or coating comprises hydrolyzable ester linkages that provide the biodegradability.

As used herein, the phrase “physiological conditions” relates to the range of chemical (e.g., pH, ionic strength) and biochemical (e.g., enzyme concentrations) conditions likely to be encountered in the intracellular and extracellular fluids of tissues. For most tissues, the physiological pH ranges from about 7.0 to 7.4. An exception is in the acidic environment in the vagina, which has a pH between 4.0 and 4.5.

As used herein, the term “supramolecular” (e.g., “supramolecular complex,” “supramolecular interactions,” “supramolecular fiber,” “supramolecular polymer,” etc.) refers to the non-covalent interactions between molecules (e.g., polymers, macromolecules, etc.) and the multicomponent assemblies, complexes, systems, and/or fibers that form as a result.

As used herein, the terms “self-assemble” and “self-assembly” refer to formation of a discrete, non-random, aggregate structure from component parts; said assembly occurring spontaneously through random movements of the components (e.g. molecules) due only to the inherent chemical or structural properties and attractive forces of those components

As used herein, the terms “prevent,” “prevention,” and preventing” refer to reducing the likelihood of a particular condition or disease from occurring in a subject not presently experiencing or afflicted with the condition or disease state. The terms do not necessarily indicate complete or absolute prevention. In order to “prevent” a disease or condition, a composition or method need only reduce the likelihood of the disease or condition, not completely block any possibility thereof. “Prevention,” encompasses any administration or application of a therapeutic or technique to reduce the likelihood of a disease developing (e.g., in a mammal, including a human). Such a likelihood may be assessed for a population or for an individual.

As used herein, the terms “treat,” “treatment,” and “treating” refer to reducing the amount or severity of a particular condition, disease, or symptoms thereof, in a subject presently experiencing or afflicted with the condition or disease. The terms do not necessarily indicate complete treatment (e.g., total elimination of the condition, disease, or symptoms thereof). “Treatment,” encompasses any administration or application of a therapeutic or technique for a disease (e.g., in a mammal, including a human), and includes inhibiting the disease, arresting its development, relieving the disease, causing regression, or restoring or repairing a lost, missing, or defective function; or stimulating an inefficient process.

As used herein, the term “administration” refers to any suitable method of providing a composition, self-assembling peptide, system, or nanofiber described herein to a subject. Administration may be by any suitable method. For example, administration may occur by directly applying to a tissue of the subject, such as directly to a wound. Suitable routes of administration include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration. In some embodiments, administration is parenteral. In some embodiments, parenteral administration is by intrathecal administration, intracerebroventricular administration, or intraparenchymal administration. The self-assembling peptides, compositions, systems, and nanofibers described herein can be administered as the sole active agent or in combination with other pharmaceutical agents such as other agents used in the treatment of a disease or condition in a subject.

As used herein, the terms “co-administration” and “co-administering” refer to the administration of at least two agent(s) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the terms “subject” and “patient” refer to any animal, such as a dog, cat, bird, livestock, and particularly a mammal, preferably a human. The subject may be male or female.

### 2. Self-Assembling Peptides

In some aspects, provided herein are self-assembling peptides. In some embodiments, provided herein are self-assembling peptides that bind to one or more components of the extracellular matrix. Suitable components of the extracellular matrix include, for example, proteins (e.g. collagen, elastin, fibronectin, laminin,), glycosaminoglycans (e.g. heparan sulfate, chondroitin sulfate, keratan sulfate), polysaccharides (e.g. hyaluronic acid), and extracellular vesicles. In some embodiments, provided herein are self-assembling peptides that bind to hyaluronic acid.

In some embodiments, the self-assembling peptides described herein are designed based upon their helical net representation. In some embodiments, the self-assembling peptides described herein are designed to achieve a desired helical net representation. The term “helical net” as used herein refers to a two-dimensional representation of tridimensional helical structures. Helical nets and how to generate the same are described in Dunnill P (1968) Biophysical journal 8:865-875, and Mól et al., “Netwheels: A web application to create high quality peptide helical wheel and net projections” bioRxiv (2018), doi: https://doi.org/10.1101/416347, the entire contents of each of which are incorporated herein by reference for all purposes. A helical net representation displays specific interactions between residues that are next to each other in the central axis of the helix. For example, intramolecular bonds between residues can be displayed. A helical net consists of parallel diagonals that are either adjacent or non-adjacent diagonals. Each diagonal consists of either even or odd numbered positions. Adjacent diagonals are diagonals where the numbered positions in one diagonal are even and the adjacent diagonal contains odd numbered positions or odd numbered and even numbered positions. In contrast, non-adjacent diagonals will have even numbered positions on both diagonals, or odd numbered positions on both diagonals.

In some embodiments, the self-assembling peptides comprise a plurality of BX₇B domains. For example, in some embodiments the self-assembling peptide comprises two, three, four, five, or six BX₇B domains. In some embodiments, the self-assembling peptide comprises four or five BX₇B domains.

The BX₇B domains may be a combination of contiguous BX₇B domains and non-contiguous BX₇B domains. The term “contiguous” refers to two B(X₇)B domains that share a common residue. The term “non-contiguous” refers to B(X₇)B domains that do not share a common residue with any other B(X₇)B domain. In some embodiments, the self-assembling peptide comprises at least two contiguous BX₇B domains and at least two non-contiguous BX₇B domains. In some embodiments, the self-assembling peptide comprises a first set of at least two contiguous BX₇B domains and a second set of at least two contiguous BX₇B domains, each set of which are non-contiguous with the other set. In some embodiments, the first and second set are on non-adjacent diagonals in a helical net representation of the peptide.

In some embodiments, the self-assembling peptides described herein are represented by helical nets containing at least two B(X₇)B domains on a first diagonal and at least two B(X₇)B domains on a non-adjacent diagonal. In some embodiments, each of the diagonals are adjacent to a diagonal that does not contain any B(X₇)B domains.

In some embodiments, the self-assembling peptide comprises at least three BX₇B domains. In some embodiments, two of the at least three BX₇B domains are contiguous and the third BX₇B domain is non-contiguous with the other two BX₇B domains.

In some embodiments, a self-assembling peptide describing a plurality of BX₇B domains as described herein can be concatenated to generate a larger self-assembling peptide.

In some embodiments, B is a basic amino acid selected from histidine, arginine, or lysine. In some embodiments, B is arginine or lysine. In some embodiments, the two “B” residues in the BX₇B domain are different. Each “B” residue is independently selected from histidine, arginine, or lysine. For example, one “B” residue may be a histidine and the other “B” residue may be an arginine. As another example, one “B” residue may be a histidine and the other “B” residue may be a lysine. Any combinations of histidine, arginine, and lysine are acceptable. In some embodiments, the two “B” residues in the BX₇B domain are the same. For example, in some embodiments both “B” residues are histidine, both “B” residues are arginine, or both “B” residues are lysine. In some embodiments, X is an amino acid selected from histidine, lysine, arginine, serine, threonine, asparagine, glutamine, alanine, valine, leucine, isoleucine, methionine, phenylalanine, tyrosine, tryptophan, proline, glycine, and cysteine. In some embodiments, the self-assembling peptide comprises one or more flanking amino acid residues. The term “flanking” indicates an amino acid that is adjacent to a “B” residue. For example, the peptide may comprise the sequence bBX₇B, bBX₇Bb, or BX₇Bb where “b” is any suitable amino acid. In some embodiments, the flanking amino acid is a positively charged amino acid (e.g. lysine, arginine, histidine, hydroxylysine, ornithine).

In some embodiments, the self-assembling peptide comprises 20-35 amino acids. In some embodiments, the self-assembling peptide comprises 20, 21, 22, 23, 24, 25, 26, 27, 28, 29, 30, 31, 32, 33, 34, or 35 amino acids. In some embodiments, the self-assembling peptide comprises 27 amino acids.

In some embodiments, the self-assembling peptide has the propensity to form β-sheet secondary structure (e.g., 3-sheet like character, such as when analyzed by CD). In some embodiments, amino acids in the self-assembling peptide are selected for their propensity to form a beta-sheet secondary structure. Examples of suitable amino acid residues selected from the twenty naturally occurring amino acids include Met (M), Val (V), Ile (I), Cys (C), Tyr (Y), Phe (F), Gln (Q), Leu (L), Thr (T), Ala (A), and Gly (G) (listed in order of their propensity to form beta sheets). However, non-naturally occurring amino acids of similar beta-sheet forming propensity may also be used. Peptide segments capable of interacting to form beta sheets and/or with a propensity to form beta sheets are understood (See, e.g., Mayo et al. Protein Science (1996), 5:1301-1315; herein incorporated by reference in its entirety).

In some embodiments, the self-assembling peptide comprises a sequence shown in Table 1. In some embodiments, the self-assembling peptide comprises a sequence selected from:

(SEQ ID NO: 1)
KTKATVLIKNKQKSKNALKQKIVLLSK,
(SEQ ID NO: 2)
LKTKIKIIVKTKSSAKLRSKLVNSHKI,
(SEQ ID NO: 3)
TQLRNKYTFLARARNALAVRTKQNIKS,
(SEQ ID NO: 4)
TNLRNKYTFLARARANLAVRNKQNIKS,
and
(SEQ ID NO: 5)
KTKATVKIKNKQKSVNALKQKIVLLSK.

In some embodiments, a plurality of self-assembling peptides described herein interact to form a nanofiber. In some embodiments, provided herein is a nanofiber comprising a plurality of self-assembling peptides as described herein. In some embodiments, provided herein is a nanofiber comprising a plurality of peptides described in Table 1. In some embodiments, the nanofiber comprises multiple types of peptides described in Table 1 (i.e., not all peptides in the nanofiber are the same).

In some aspects, provided herein are systems. In some embodiments, provided herein is a system comprising a self-assembling peptide described herein bound to a component of the extracellular matrix. Suitable components of the extracellular matrix include, for example, proteins (e.g. collagen, elastin, fibronectin, laminin,), glycosaminoglycans (e.g. heparan sulfate, chondroitin sulfate, keratan sulfate), polysaccharides (e.g. hyaluronic acid), and extracellular vesicles. In some embodiments, provided herein is a system comprising a self-assembling peptide as described herein bound to collagen, elastin, or hyaluronic acid. In some embodiments, the self-assembling peptide is bound to hyaluronic acid. Hyaluronic acid refers to a repeating disaccharide polymer, also referred to as hyaluronan, often found in vertebrate tissues as a key component of the extracellular matrix. Hyaluronic acid (HA) is a linear anionic non-sulfated glycosaminoglycan composed of two alternating units, D-glucuronic acid and N-acetyl-D-glucosamine, linked by alternating glycosidic bonds (β-(1→4) and β-(1→3). Hyaluronic acid is represented by the formula (C₁₄H₂₁NO₁₁)n. The molecular weight of hyaluronic acid depends on the number of repeating disaccharides present in the molecule. The molecular weight of hyaluronic acid can range from 1,000 to millions of kD, depending on the source in which it is found. In some embodiments, hyaluronic acid has a molecular weight of 1000-8000 kDa. Such a mass is referred to as a “high” molecular weight hyaluronic acid. As used herein, the term “low” molecular weight hyaluronic acid refers to hyaluronic acid having a molecular weight of less than 1,000 kDa (e.g. 8700-900 kDa), whereas a “high” molecular weight hyaluronic acid refers to hyaluronic acid having a molecular weight of at least 1,000 kDa. Low or high weight hyaluronic acid may be used here, although in some embodiments high molecular weight hyaluronic acid may be preferred, such as for methods of promoting wound healing.

In some embodiments, self-assembling peptides bound to hyaluronic acid will form a gel-like structure, such as a hydrogel. Such a hydrogel may be formed as a result of non-covalent crosslinking of nanofibers. In some embodiments, to induce self-assembly of an aqueous solution of peptides, the pH of the solution may be changed (raised or lowered) or multivalent ions, such as calcium, or charged polymers or other macromolecules may be added to the solution. In some embodiments, beta-sheet formation may be induced by increasing the concentration (e.g. amount) of self-assembling peptides in a solution. For example, increasing concentrations of self-assembling peptides may facilitate formation of beta-sheet structures, thus facilitating formation of nanofibers (e.g. ribbons) or formation of a hydrogel in the presence of hyaluronic acid.

In some embodiments, the self-assembling peptides or nanofibers described herein may be incorporated into a composition. In some embodiments, the self-assembling peptides, nanofibers, compositions, and systems described herein find use in various methods.

In some embodiments, the self-assembling peptides, nanofibers, compositions, and systems described herein find use as a coating for a material. For example, provided herein is a material coated with a plurality of self-assembling peptides as described herein, such as a plurality of self-assembling peptides that bind to a component of the extracellular matrix (e.g. hyaluronic acid). In some embodiments, provided herein is a material coated with a system comprising a plurality of self-assembling peptides bound to a component of the extracellular matrix, such as hyaluronic acid. The material may be a biomedical device. The term “biomedical device” refers to any suitable biocompatible device designed for use in a human subject, such as for implantation or insertion into a human subject for a medical purpose. In some embodiments, the material comprises a neural implantable device. The term “neural implantable device” refers to any device that may be implanted into the central nervous system or peripheral nervous system of the subject, including the brain, spinal cord, or nerves. For example, a neural implantable device may be any suitable device for deep brain stimulation, an intraspinal simulator, a microstimulator, a neural chips, etc. The use of such a coated device may mediate neural inflammation following implantation of the device, and may thus help facilitate prevention of rejection by the subject. In some embodiments, the material is a surgical material. For example, the material may be a surgical material such as suture material, staples, gauze, bandages, etc. In some embodiments, the material is a wound dressing (e.g. gauze, bandage, etc.). The use of such a coated wound dressing or surgical material may be used to promote wound healing, including following surgery or after otherwise sustaining an injury or illness causing a wound.

In some embodiments, the self-assembling peptides, nanofibers, compositions, and systems described herein find use as a coating for a biomaterial. For example, the self-assembling peptides, nanofibers, compositions, and systems described herein may be used for lubricating a biomaterial, such as a joint, a tissue, or an organ. For example, hyaluronic acid is a cushion and lubricant for joints and tissues, including the eyes. Accordingly, a system comprising a self-assembling peptide bound to hyaluronic acid may be administered to a subject in need thereof to promote lubrication of a joint, a tissue, or an organ in the subject.

In some embodiments, the self-assembling peptides, nanofibers, compositions, and systems described herein find use in methods of treating various conditions including inflammatory conditions, cancer, and promoting wound healing in a subject.

In some embodiments, the self-assembling peptides, nanofibers, compositions, and systems described herein find use in methods of treating an inflammatory condition in a subject. For example, the self-assembling peptides, nanofibers, compositions, and systems described herein may be provided to a subject in need thereof to treat an inflammatory condition such as lung inflammation (e.g. as a result of a condition such as asthma, bronchitis, pneumonia, a viral infection, fungal infection, a bacterial infection, and the like.). As another example, the self-assembling peptides, nanofibers, compositions, and systems described herein may be used in a method of treating an inflammatory condition such as arthritis (e.g. rheumatoid arthritis, osteoarthritis).

In some embodiments, the self-assembling peptides, nanofibers, compositions, and systems described herein find use in methods of treating cancer in a subject. The terms “cancer” and “carcinoma” refer to or describe the physiological condition in mammals that is typically characterized by unregulated cell growth. The pathology of cancer includes, for example, abnormal or uncontrollable cell growth, metastasis, interference with the normal functioning of neighboring cells, release of cytokines or other secretory products at abnormal levels, suppression, or aggravation of inflammatory or immunological response, neoplasia, premalignancy, malignancy, invasion of surrounding or distant tissues or organs, such as lymph nodes, blood vessels, etc. The cancer may be any cancer type.

In some embodiments, the self-assembling peptides, nanofibers, compositions, and systems described herein find use in methods of treating a wound in a subject. Hyaluronic acid (HA), a component of extracellular matrix, thought to modulate tissue regeneration along with phases of wound healing including inflammation, cell migration, and angiogenesis. In particular, high molecular weight HA displays anti-inflammatory and immunosuppressive properties, whereas low molecular weight HA is a potent proinflammatory molecule. The self-assembling peptides described herein may promote scarless wound healing. For example, the self-assembling peptides, nanofibers, compositions, and/or systems described herein may be applied directly to the site of the wound in a subject, thereby promoting wound healing in the subject. As another example, the self-assembling peptides, nanofibers, compositions, and/or systems described herein may be used as a coating for a wound dressing that is applied to the wound.

Embodiments of the disclosed composition are set forth in the following non-limiting examples.

### EXAMPLES

### Example 1

### Materials & Methods

### Materials

Biotinylated peptides were custom synthesized by ABI Scientific, Inc. (Sterling, VA). Uranyl acetate and TEM grids were from Ted Pella, Inc (Redding, CA). Unless specified otherwise, all water used in these experiments was Milli-Q grade. HA (˜1 MDa), collagen types I (rat), II (chicken), III (human), and IV (mouse), elastin (bovine), deuterium oxide (D₂O), acetic acid-d4, sodium acetate-d3, 3-(Trimethylsilyl)propionic-2,2,3,3-d6 (DSS-D₆) acid sodium salt, and 2,2,2-trifluoroethanol (TFE) and were obtained from Sigma-Aldrich (St. Louis, MO). Streptavidin (Alexa Fluor 488 conjugate) and Geltrex were obtained from Thermo Fisher Scientific Inc. (Waltham, MA). Sodium hyaluronate (5 and 60 KDa) was purchased from Lifecore Biomedical LLC (Chaska, MN). In reference to terminology, N and C termini is used to describe the amino terminal (N) and the carboxyl terminal (C) of the peptide. The biotinylated peptides are used in every experiment.

### Peptide Binding and Specificity Testing

Hyaluronic acid, collagens I, II, III, and IV, elastin, and Geltrex were coated onto 96-well black plates (medium binding affinity) at 10 μg/cm² and were dried in milli-Q water at room temperature. N-termini biotinylated peptides were added to coated plates at 0, 1, 2.5, 5 and 10 mg/ml overnight at 37° C. in phosphate buffered saline (PBS), pH 7.4, after which the plates were washed three times with PBS. Fluorescent streptavidin-488 (4 μg/mL) was added to each well and incubated at 37° C. temperature for 24 hours, then thoroughly washed with PBS. The plates were read with a fluorescence microplate reader for fluorescence intensity (Ex 490 nm, Em 525 nm). All data were normalized to the background fluorescence intensity of Streptavidin-488.

### Nuclear Magnetic Resonance (NMR)

Peptides and HA were suspended in 25 mM sodium acetate buffer (pH 5.2), made up from 19 mM sodium acetate-d3, 6 mM acetic acid-d4, 10% D₂O, and 1 mg/ml DSS-D₆. All samples were centrifuged at 21,000 G to remove precipitate and the supernatant was collected for analysis, as only the liquid suspension could be used. NMR spectra were collected on an Avance NEO 600 MHz spectrometer at 298K with excitation sculpting used to suppress the water signal. Number of scans and receiver gain were identical for all NMR spectra.

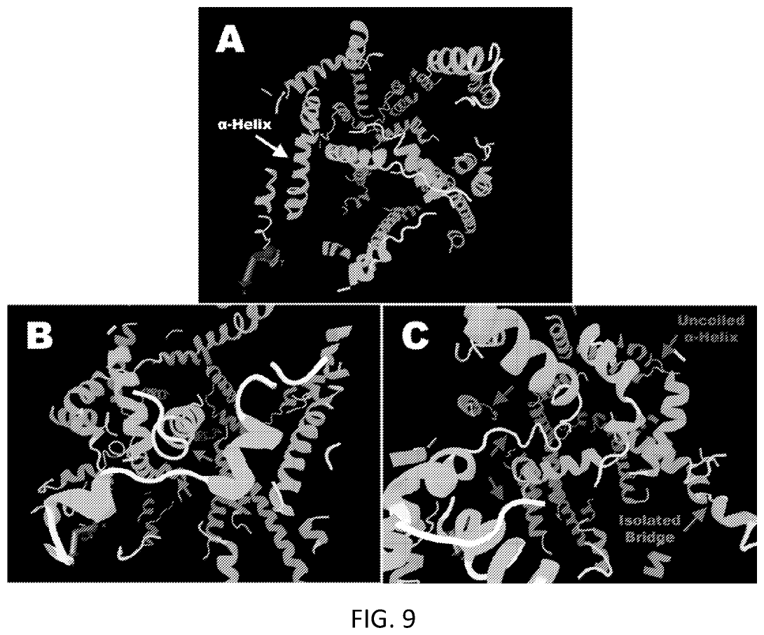

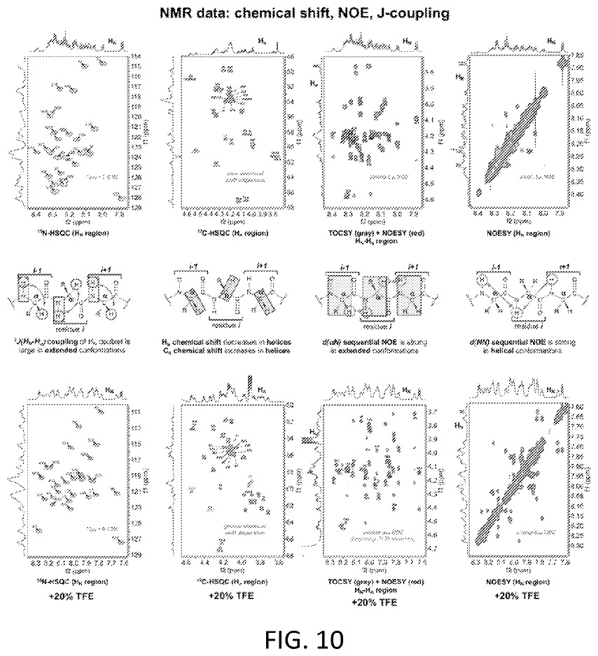

Analysis of secondary conformations were performed with 2D spectra of chemical shift, using nuclear Ovehauser effect (NOE), and J-coupling of peptide 17x-3 at 5 mg/ml, in sodium acetate buffer (pH 5.4), without and with 20% TFE. Spectra and assignments include ¹⁵N heteronuclear single quantum coherence (¹⁵N-HSQC), ¹³C-HSQC, total correlation spectroscopy (TOCSY)/NOESY H_{N}-H_{α} regions, and NOESY H_{N} regions. Examples are including in the figure (FIG. 9) of which protons in carbonyls and NHs were selected per spectra. TALOS-N chemical shift analysis was then performed of 17x-3 based 2D spectral assignments of 17x-3.

### Circular Dichroism (CD)

Peptides were sonicated for 30 minutes at concentrations 1.25, 2.5, and 5 mg/ml, and incubated at 37° C. for 24 hours before peptides were pipetted into a 0.5 mm type 20 demountable 0-shaped quartz cuvette. Sodium cacodylate (SC), 20 mM pH 7.4, without and with 20% TFE was used to reduce phosphate related background signal and enhance detected structures. Circular dichroism (CD) data were collected in millidegrees, 0, and converted to molar ellipticity, [θ]_{M}, and are expressed in units of deg×cm²×dmol⁻¹. Scan speeds and digital integration times were 500 nm/min and 1 second, respectively. The data on the Y axis of the CD plots is [θ]_{M}×10⁻³. All CD spectra were collected using a Jasco J-815 CD spectrophotometer. Based on the fact that experiments were conducted at increasing concentrations of peptide it was anticipated that intermolecular interactions could potentially result in aggregates, thus the samples were not syringe filtered because this procedure could potentially remove the peptide of interest. Consequently, CD signals are reported in the 190-260 nm range. HTV (High Tension Voltages) values were well within the 700 volt limit specified by the manufacturer, with the exception of three values at 190 nm. Molar concentrations are provided in the Table 5.

### Transmission Electron Microscopy (TEM)

All protocols were followed from Dawes et al. (Dawes, C. J. Biological Techniques for Transmission and Scanning Electron Microscopy, [2d ed.]; Burlington, Vt.: Ladd Research Industries, 1979.) Peptide samples were pipetted from the center of the sample container, inserted onto perforated formvar carbon coated copper grid and a 4% uranyl acetate stain applied. The sample and stain were filter-dried in between steps. Samples were collected 24 hours at 37° C. after 30 minutes of sonication. All TEM was performed on a Philips FEI Technai Spirit G2 electron microscope. Peptides were incubated in both PBS and SC.

### Fourier Transform Infrared (FTIR)

Fourier transform infrared spectroscopy was performed on a Thermo Fisher Scientific Nicolet IS50R FT-IR using a deuterated triglycine sulfate (DTGS) detector in the CBC Keck Center for Nano-Scale Imaging. Peptide samples were dissolved in either 20 mM sodium cacodylate or in sodium cacodylate containing 20% trifluoroethanol. Data are reported in wave numbers (cm⁻¹), to the nearest wave number. Scans are reported in the amide I region (1700-1600 cm⁻¹) and in the amide A region (˜3300 cm⁻¹) or (3310-3270 cm⁻¹). The original scans were deconvoluted using the Fourier Self-Deconvolution (FSD) methodology (K. Kauppinen, D. J. Moffatt, H. H. Mantsch, D. G. Cameron, Fourier Self-Deconvolution: A Method for Resolving Intrinsically Overlapped Bands, Appl. Spectrosc., AS. 35 (1981) 271-276), which resolves overlapping bands in the FTIR through a “band narrowing procedure”. For any scans that were still unresolved, the scans were reanalyzed at a higher sensitivity. Antiparallel β-sheets have two resonance transition regions designated B1 and B2. The B1 region has an average value of 1696 cm⁻¹ and ranges from 1705-1685 cm⁻¹. The B2 region has an average value of 1629 cm⁻¹ and ranges from 1637-1615 cm⁻¹. Lastly, the wave numbers have been stated from high to low wave number based on IUPAC convention (A. Barth, Infrared spectroscopy of proteins, Biochim Biophys Acta. 1767 (2007) 1073-1101. https://doi.org/10.1016/j.bbabio.2007.06.004.)

### Size Exclusion Chromatography Multiangle Light Scattering and Quasi-Elastic Light Scattering (SEC-MALS-QELS)

Size Exclusion Chromatography Multiangle Light Scattering and Quasi-Elastic Light Scattering (SEC-MALS-QELS) was performed by the Keck Biophysics Facility at Northwestern University. A Wyatt DAWN HELEOS II multi-angle detector, a Wyatt Optilab T-rEx refractometer, a Wyatt QELS quasi-elastic light scattering detector, and a high pressure liquid chromatographic (HPLC) system with a diode array detector were used. A scattering wavelength of 658 nm was chosen, each sample was standardised to bovine serum albumin in PBS, with flow rates of 0.4 mL/min.

### In Silico Modelling

GROMACS 4.6.5 was used to simulate the molecular dynamics of 17×3 in solution. The GROMOS 53a6 force field was selected when generating topologies. The system was solvated with SPC water molecules in a 512 cubic nanometer box. The system was then neutralized with counter ions. A 1000-step steepest descent energy minimization was applied to the system. Then, the simulation box was equilibrated at a reference temperature of 300 K and a reference pressure of 1 bar using the NVT and NPT ensembles. Pressure coupling was maintained with an isotropic Parrinello-Rahman barostat while temperature was maintained with a V-rescale modified Berendsen thermostat. The Verlet cut-off scheme with a short range cut-off radius of 1.2 nm was used. Holonomic constraints were set using the LINCS algorithm. Long-range electrostatic interactions were calculated through Particle Mesh Ewald method using fourth order cubic interpolation and 0.16 nm grid width. A 50 ns production run was executed with all position restraints removed. The B1 region has an average value of 1696 cm⁻¹ and ranges from 1705-1685 cm⁻¹.

### Statistical Analyses

Overall significance was assessed using analysis of variance, with Tukey's multiple comparison post-hoc analysis between peptide groups. Pairwise comparisons were assessed using a Mann-Whitney U test. A p value of ≤0.05 was considered significant. Data are presented as the mean±SEM. Each reported n indicates an independent experiment.

### Results

### Overview

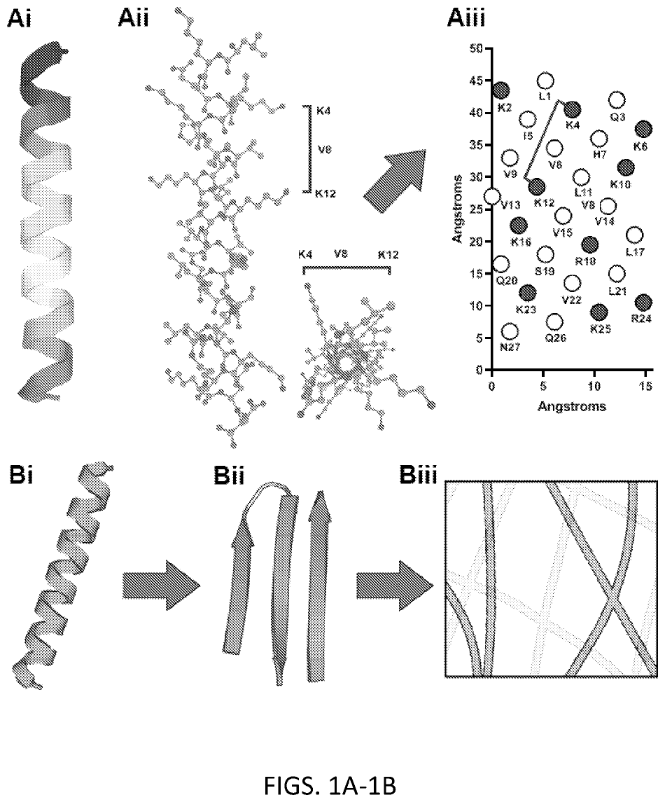

Provided herein are hyaluronic acid binding peptides that are self-assembling. Ten peptides were prepared with combinations of contiguous and non-contiguous hyaluronic acid binding domains, all of which were shown to form beta-sheets and six self-assemble into two different types of nanofibers. The designed peptides, with respective sequences, are presented in Table 1. The designed peptides were tested for concentration and extra-cellular matrix dependent binding, peptide structure was assessed using circular dichroism (CD), and higher order nanostructural self-assembly was characterized using transmissions electron microscopy (TEM). For this novel class of peptides, the relationship between design, binding, and peptide biophysics was determined. FIG. 1 shows an overview of an exemplary process for peptide design and evaluation of peptide binding and biophysics.

### Design of Peptides

Peptides containing B(X₇)B domains that bind hyaluronic acid (HA) were designed, where B is any basic amino acid and X is any amino acid, except an acidic amino acid. HABP35 was prepared by covalently linking RHAMM binding domain I to RHAMM binding domain II and was designed to have four B(X₇)B domains (Zaleski et al., Hyaluronic Acid Binding Peptides Prevent Experimental Staphylococcal Wound Infection. Antimicrob Agents Chemother 2006, 50 (11), 3856-3860. https://doi.org/10.1128/AAC.00082-06) A peptide termed mPEP35 was used as a reference peptide.

Table 1 reports the design of the peptides, including the amino acid sequence, configuration of B(X₇)B binding domains (contiguous or non-contiguous, based on the helical net), number of residues and a hydrophobicity/hydrophilicity ratio. Contiguous was defined as two B(X₇)B domains that share a common residue and non-contiguous as those B(X₇)B domains that do not share a common residue with any other B(X₇)B domain. mPEP35 has two contiguous and two non-contiguous B(X₇)B domains. Peptide 1-scrm is a scrambled sequence of mPEP35, in a way that contains no B(X₇)B domains and is used as a negative reference in the present study. Furthermore, the composition of hydrophobic and hydrophilic amino acids in mPEP35 results in a hydrophobic to hydrophilic ratio of 1:1.3. The scale of Monera et al. was used to calculate the hydrophobicity/hydrophilicity ratio (Monera et al., Relationship of Sidechain Hydrophobicity and Alpha-Helical Propensity on the Stability of the Single-Stranded Amphipathic Alpha-Helix. J. Pept. Sci. 1995, 1 (5), 319-329). The same characteristics for all other peptides are reported in Table 1. In addition, the conformation of exposed B(X₇)B domains were plotted using a helical net rule drawn according to Sereda et al. (Sereda et al., Reversed-Phase Chromatography of Synthetic Amphipathic Alpha-Helical Peptides as a Model for Ligand/Receptor Interactions. Effect of Changing Hydrophobic Environment on the Relative Hydrophilicity/Hydrophobicity of Amino Acid Side-Chains. J Chromatogr A1994, 676 (1), 139-153).

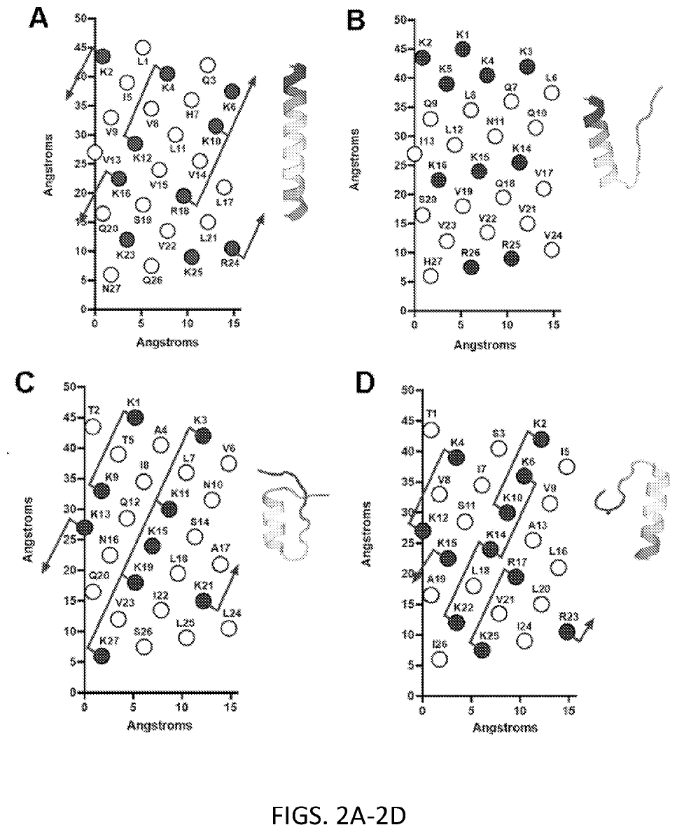

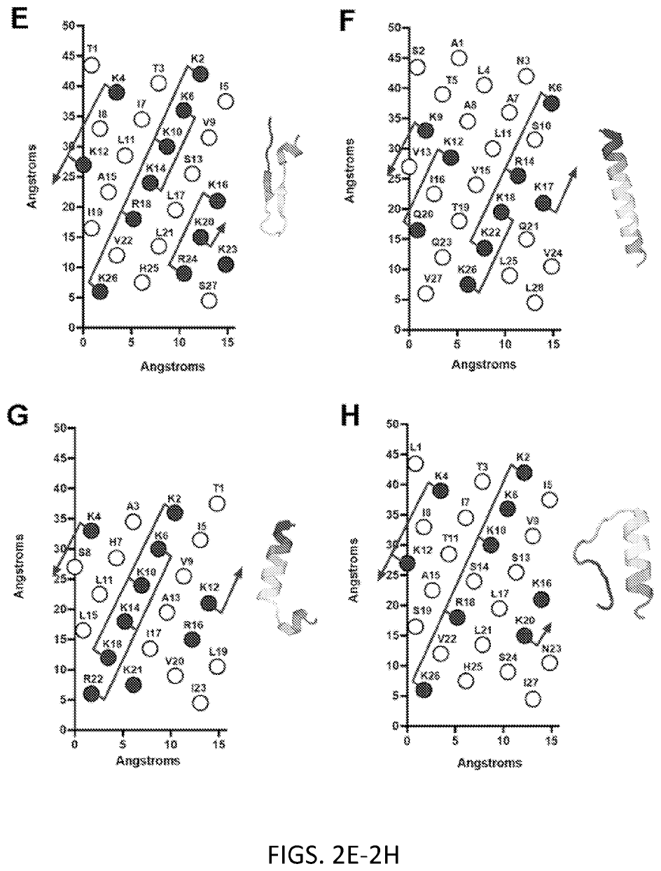

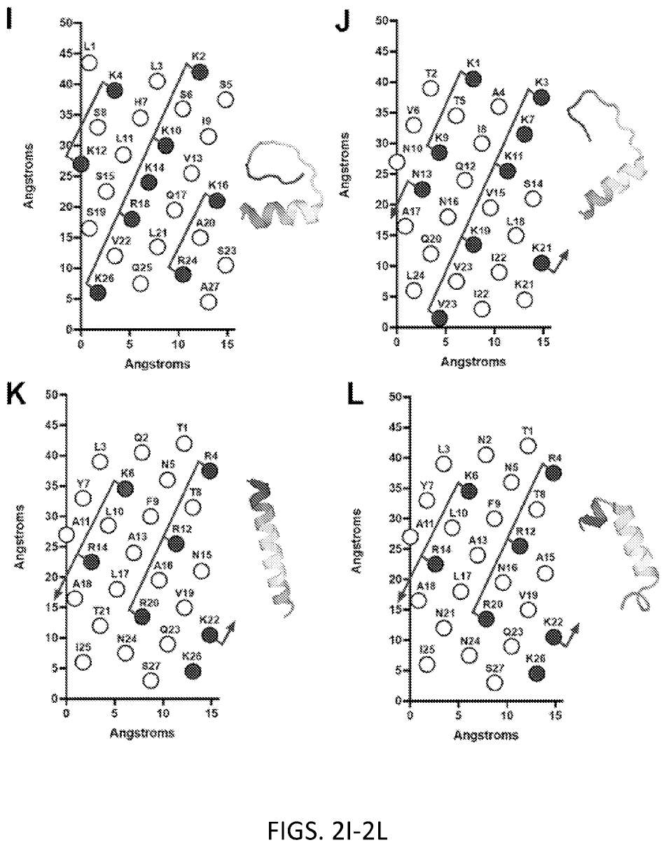

Example helical nets and predicted PEP-FOLD models are shown in FIG. 2. The position of the α-carbon atom is depicted as a circle on the plot and is based on the α-helix being a cylindrical surface where there are 3.6 residues per turn and a 1.5 Å translation for each amino acid along the helix. The two dimensional plot represents a cylindrical surface where the width of the net represents the circumference of the cylinder (15.7 Å), based on the alpha helix having a radius of 2.5 angstroms. Proposed contiguous or non-contiguous B(X₇)B domains can be designed into the peptide net, with a upward slope where two positive charged amino acids (lysine/arginine) are spaced by any other residue (see blue lines), except a negatively charged residue.

Peptide 17x-3 is a de novo designed peptide, based on the following criteria—mPEP35 has: (i) four B(X₇)B domains, (ii) has 27 residues and (iii) has contiguous and non-contiguous B(X₇)B domains. Peptide 17x-3 is designed to have five B(X₇)B domains within 27 residues and in a different configuration than in mPEP35, i.e., three contiguous and two non-contiguous B(X₇)B domains. Furthermore, the composition of hydrophobic and hydrophilic amino acids for 17x-3 is designed to be similar to mPEP35, i.e., 17x-3 has a hydrophobic to hydrophilic ratio of 1:1.3. Peptide 6f-2 and 8h-2 are modifications of mPEP35 and are designed to have either six or seven B(X₇)B domains in order to determine the effect of HA binding. Peptide 2^{nd} de novo has five B(X₇)B domains and peptide 10K is designed to have five B(X₇)B domains in a span of 23 residues.

The following peptides are variants: (i) peptide 4 is a variant of peptide 8h-2, (ii) peptide 6b is a variant of peptide mPEP35 (iii) peptide 7c is a variant of peptide 17x-3 and has the same five B(X₇)B domains as 17x-3 and (iv) BHP3 and BHP4 are designed from BH-P¹⁴ to have 27 residues and four B(X₇)B domains.

### Hyaluronic Acid Binding

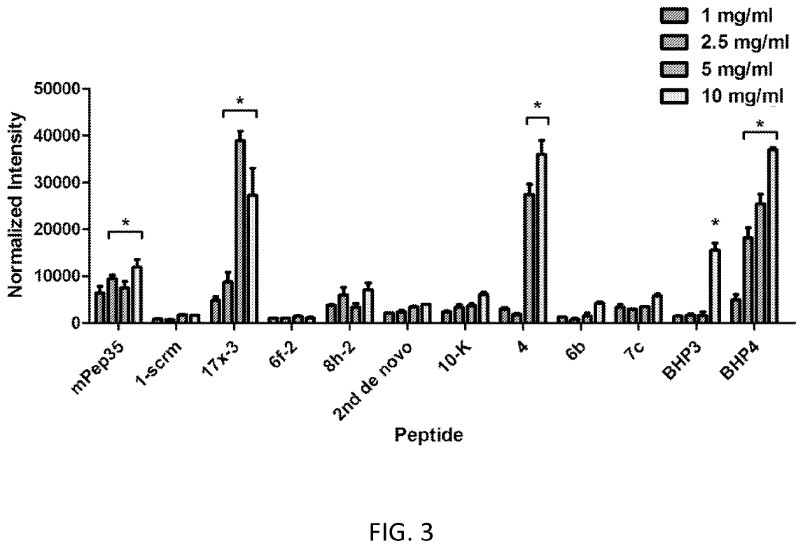

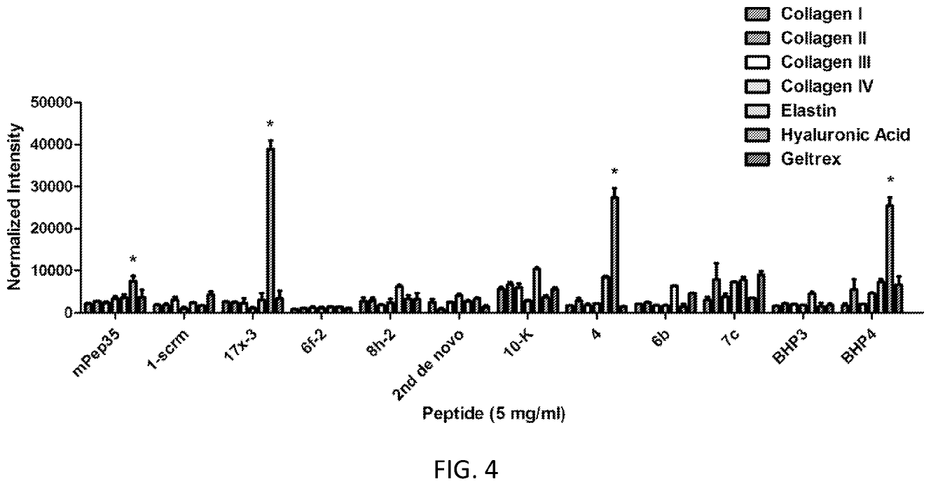

FIGS. 3 and 4 demonstrate the binding of the test peptides to extracellular matrix (ECM) components: Collagen type I, II, III and IV, elastin, hyaluronic acid and Geltrex. From FIG. 3, it can be seen that mPEP35 binds to hyaluronic acid moderately with increasing concentrations of peptide; whereas, the negative reference peptide demonstrates little to no binding to hyaluronic acid with increasing concentrations of peptide. Four peptides from FIG. 3, i.e., peptides 17x-3, 4, BHP3 and BHP4, demonstrate binding to hyaluronic acid that increases dramatically with increasing concentrations of peptide: (i) 17x-3 and 4 show increased binding at 5 and 10 mg/ml, (ii) BHP3 shows increased binding at 10 mg/ml and (iii) BHP4 shows increased binding at 2.5, 5.0 and 10 mg/ml. The other peptides show significantly lower binding to hyaluronic acid, relative to mPEP35 and one peptide, i.e., 6f-2, shows binding that is no different than the negative reference peptide. It should be noted that for 17x-3, 4, BHP3 and BHP4, a layer appeared to form on top of the HA coating and could be peeled back like a piece of tape, suggesting that the peptides formed some type of gel like structure.

Specificity was evaluated by testing the ability of the peptides to bind to various ECM components. From FIG. 4, it can be seen that three of the four peptides that demonstrated increased binding to hyaluronic acid with increasing concentrations of peptide, also demonstrated various levels of binding to the ECMs. Peptide 17x-3 binds to ECMs with high specificity, i.e., the peptide binds primarily to hyaluronic acid and does not bind significantly to the other ECMs. Peptide 4 binds with lower specificity than 17x-3, i.e., peptide 4 binds predominantly to hyaluronic acid and moderately to elastin. Peptide BHP4 binds to the ECMs with the lowest specificity, i.e., BHP4 not only binds to hyaluronic acid, but binds moderately to collagen II, IV, elastin and Geltrex. It should be noted that peptide BHP3, which bound significantly to hyaluronic acid at 10 mg/ml, did not show significant binding to the ECMs, i.e., binding to the ECMs was at a level similar to the negative reference. It is worth noting that BHP3 demonstrated significant binding to hyaluronic acid at 10 mg/ml peptide, whereas the specificity testing was done at 5.0 mg/ml. It is also noteworthy to point out that there are two other peptides that demonstrated moderate binding to ECMs; although, with lower specificity than either 17x-3 or peptide 4. Peptide 10K and 7c bind with similar specificity to BHP4. Peptide 10K binds predominantly to collagen type I, II, III and elastin; whereas, peptide 7c binds predominantly to collagen type I, IV and elastin. Thus, even though the specificity is low for these two peptides, each peptide demonstrates unique binding to specific ECMs, which may provide an advantage in specific applications.

### Circular Dichroism of Peptides

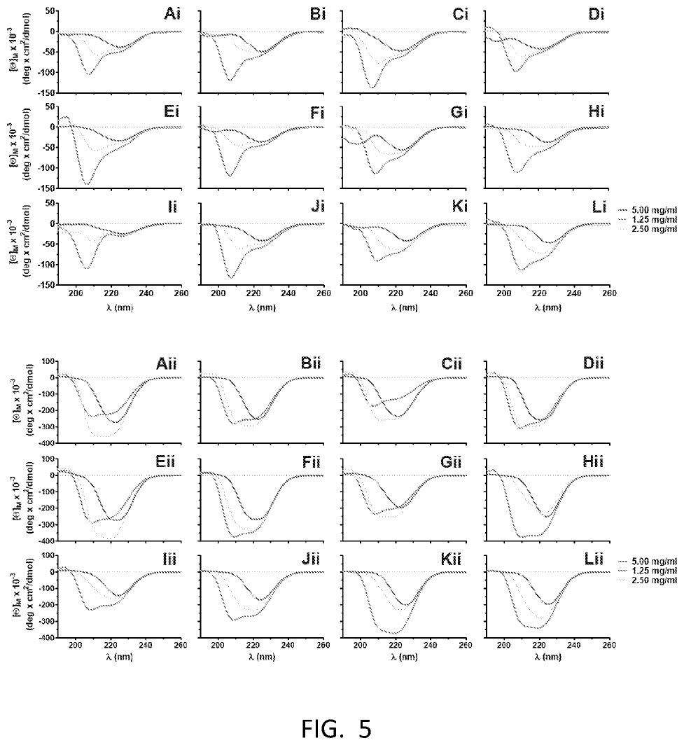

Since some of the peptides in the hyaluronic acid binding assay demonstrated increasing levels of binding with increasing concentration, CD experiments were done to determine the effect of concentration on secondary structure. FIG. 5 show the circular dichroism (CD) profiles for mPEP35, 1-scrm, 17x-3, 6f−2, 8h−2, 2^{nd} denovo, 10K, 4, 6b, 7c, BHP3, and BHP4 in either 20 mM sodium cacodylate (e.g., FIG. 5Ai for mPEP35) or in 20 mM sodium cacodylate and 20% TFE (e.g., FIG. 5 Aii for mPEP35). From the CD profiles of mPEP35 at 1.25 mg/ml in 20 mM sodium cacodylate, there is a minimum at less than 210 nanometers, at 2.5 mg/ml there is a minimum at greater than 210 and at 5.0 mg/ml there is minimum at greater 220 nanometers. Additionally, the CD profiles are considerably different from each other. These two observations suggest that the secondary structure is changing, due to increases in concentration of the peptide and a similar effect is observed in 20 mM sodium cacodylate/20% TFE. In fact, this effect is observed for all the peptides tested. As well, in sodium cacodylate, the profiles exhibit a major band near 210 nanometers and a shoulder at about 222 nanometers, thus, [θ]_{222 nm}/[θ]_{208 nm} ratios were determined for all the peptides and examination of the four target peptides identified in the hyaluronic acid binding assay demonstrated a range of[θ]_{222 nm}/[θ]_{208 nm} ratios: 17x-3 is 0.48 and peptide 4 is 0.54, compared to BHP3 (0.79) or BHP4 (0.81) in sodium cacodylate. A range of [θ]_{222 nm}/[θ]_{208 nm} ratios below 0.85 is observed for all the peptides in sodium cacodylate, suggesting that none of the peptides are fully helical. As well, the peptides have one profile in sodium cacodylate and a different profile in sodium cacodylate/20% TFE, suggesting that the TFE is causing a conformational change in the peptides. Furthermore, addition of TFE to the peptides in sodium cacodylate causes an increase in band intensity (i.e., more negative), suggesting there is more secondary structure than in sodium cacodylate alone. Lastly, all the peptides in 5.0 mg/ml buffer demonstrate one CD band at a wavelength that is greater than 220 nanometers, either in sodium cacodylate alone or in sodium cacodylate/20% TFE.

### Nuclear Magnetic Resonance (NMR) of Peptide-Hyaluronic Acid Complexes

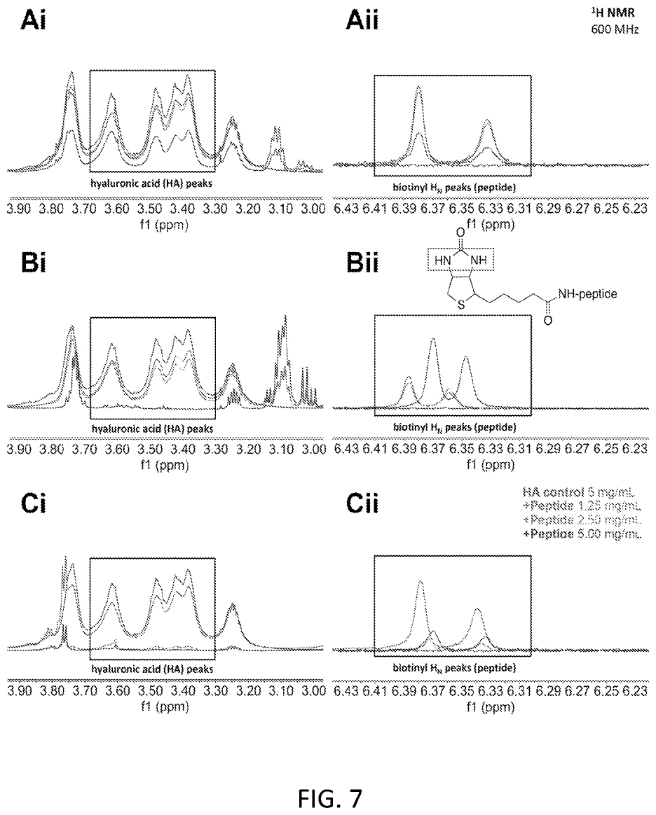

As higher peptide binding was accompanied by apparent gel-like structure on the substrate, the binding event may be characterized by the change in concentration of either peptide or HA remaining in the solution. ¹H NMR is an ideal method for measuring changes in concentration in solution. It provides atom-specific information that can be used to distinguish signals of biotinylated peptides (6.4 ppm) from those of hyaluronic acid (3.5 ppm) as presented in FIG. 7. Sodium acetate (pH 5.2) was chosen as the buffer since it provides better spectral quality of the amide signals than pH 7.4 buffer. Focus was placed on characterizing the binding of mPEP35, 1-scrm, and 17x-3, which reflect a positive control, a negative control, and a potent binder, respectively. Measurements were taken after centrifugation to remove gel that formed when peptide and HA are mixed. It should be noted that only H_{N} peaks of the peptide show slight chemical shift perturbation upon addition of HA. The alpha protons of the peptide (4-5 ppm) have the same chemical shift with and without HA, indicating that peptides remaining in solution are always in random coil conformation.

When compared to spectra of pure peptide, mPEP35 HA peaks are reduced approximately to 20% of with addition of 1.25 and 2.5 mg/mL peptide, and 40% (area under the curve) is lost with 5 mg/mL. When 5 mg/mL of the control 1-scrm peptide is introduced, ˜50% peptide remains while the HA spectra is nearly baseline. The HA losses due to the addition of 17x-3 peptide are more profound; the spectras are nearly gone at 2.5 and 5 mg/mL with only ˜80% being retained with 1.25 mg/mL peptide. It should be noted that 5 mg/mL may be a concentration for peptides mPEP35 and 1-scrm that allows for the visually observed complexes to remove portions of HA from solution, and that this shift may have a lower concentration threshold for the 17x-3 peptide shift as the low peptide concentration 1.25 mg/mL retained the only HA spectra of note. Furthermore, these concentrations for the loss of HA spectra also coincide with ppm peak shifts which could be indicative of solvent effects due to lost HA, not discernable changes in peptide structure. The percent lost of the peptide peaks is clearly not linear compared to the peptide concentration, suggesting that significant portions of peptide participate in forming these complexes. This is especially noteworthy at the highest concentration 5 mg/ml for mPEP35 and 17x-3.

### TEM

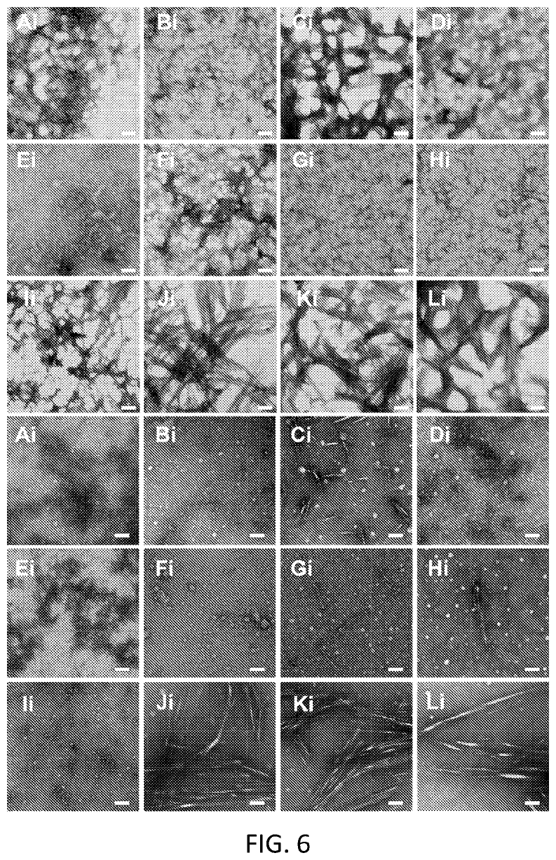

FIG. 6 shows the TEM micrographs for mPEP35, 1-scrm, 17x-3, 6f−2, 8h−2, 2^{nd} de novo, 10K, (H) 4, 6b, 7c, BHP3, and BHP4 in either phosphate buffered saline (PBS) (e.g., FIG. 6Ai for mPEP35) or in 20 mM sodium cacodylate (e.g., FIG. 6Aii for mPEP35). Additionally, Table 3 documents the observations in the TEM. From the micrographs it can be seen that four peptides give consistent nanofibers, i.e., peptides 17x-3, 4, BHP3 and BHP4 in both PBS and sodium cacodylate. The general observation is the peptides in phosphate buffered saline (PBS) demonstrate fibers that are considerably different from the fibers in 20 mM sodium cacodylate, i.e., in PBS, fibers are composed of a few too many striations (fibrils); whereas, in 20 mM sodium cacodylate, the fibers are fewer in number and appear to be a twisted ribbon (Paired 3 sheet structure).²² The exception is mPEP35, i.e., there are fibers containing a small number of striations (2 fibrils mostly and up to 5 in some cases) that go to form larger fibers in PBS; whereas, in20 mM sodium cacodylate, no fibers or structure can be observed. It should be noted that peptide 7c demonstrates micrographs that are similar to 17x-3, 4, BHP3 and BHP4.

For the four peptides that demonstrated significant binding in the HA binding assay, the following is observed. For peptide 17x-3 in PBS, fibers with many striations form larger fibers and the larger fibers tend to form a mesh network; whereas, in 20 mM sodium cacodylate, fibers appear to be a twisted ribbon. For peptide 4 in PBS, there appears to be fibers that contain two striations (fibrils) and these two stranded fibers either forma network or can form a mesh of larger sized fibers; whereas, in 20 mM sodium cacodylate, there appears to be three twisted ribbon fibers, that are adjacent to each other. BHP3 and BHP4 produce very similar results, i.e., in the presence of PBS, both peptides form fibers with many striations that form larger fibers and the larger fibers tend to form a mesh network. Alternatively, in 20 mM sodium cacodylate, BHP3 and BHP4 form apparent twisted ribbons.

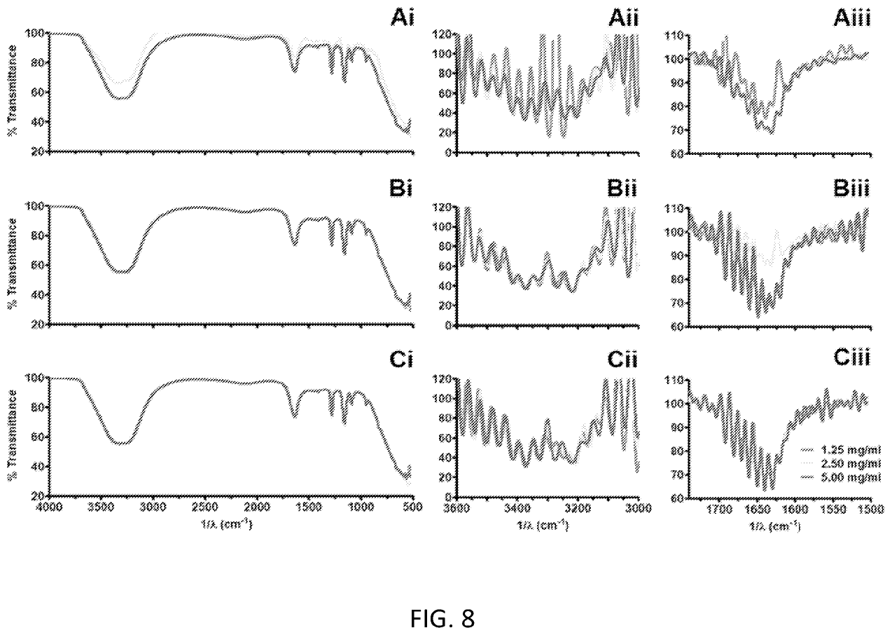

Fourier Transform Infrared Spectroscopy (FTTR) of Peptides Representative FTIR spectra and deconvolutions are shown in FIG. 8 and a summary of peaks of interest are presented in Table 4 for peptides in 20% TFE. Peaks measured without TFE are summarized in Table 6. Table 4 also footnotes are the ranges of wave numbers obtained from the literature that were utilized to identify specific secondary/tertiary structures. Since the CD data indicated the presence of 3₁₀-helices (in SC buffer only), α-helices in SC/20% TFE and β-sheet (in both experimental conditions), FTIR analysis was performed to confirm these observations. The data in SC buffer and SC buffer/20% TFE demonstrates that these structures are indeed present in the peptides analysed. More specifically in the range 1665-1655 cm⁻¹ for 3₁₀-helices [36], and 1660-1648 cm⁻¹ for α-helices [52]. The FTIR data also indicate the presence of an α-/3₁₀ helix conformation at 1673-1670 cm⁻¹ (also called mixed α/3₁₀-forms, which contain hydrogen bonding characteristics consistent with α-helices and 3₁₀-helices [49,53,54]). Two distinct signatures at ≈1695-1675 cm⁻¹ and ≈1638-1632 cm⁻¹ or 1640-1610 cm⁻¹ [52] signify an antiparallel β-sheet [36]. More specifically, i.e., 1685, 1641 and 1636 cm⁻¹[55] for intramolecular β-sheets and (ii) 1695 and 1625, 1625-1615 cm⁻¹[56] or 1692 and 1625 cm⁻¹[55] for intermolecular β-sheets.

### In Silico Modelling of Peptides

To complement the experimental findings of these self-assembling peptides in silico modelling was used to simulate the molecular dynamics of 17x-3 in solution. This is shown in FIG. 9. In the initial production run, many of the 17x-3 peptides begin to interact and uncoil from their α-helical structure. A small number of proteins formed isolated bridges, a precursor to the formation of β-sheets. This is shown in FIGS. 9b and c. This prompted the undertaking of another set of simulations, which are currently in progress. The next goal is to increase protein concentration in the simulation box to examine the complete shift of the peptides from α-helices to β-sheets. Further experimentation with TFE and HA to elicit greater β-sheet formation is also being attempted.

### 2D NMR Assignments of Peptide Structure

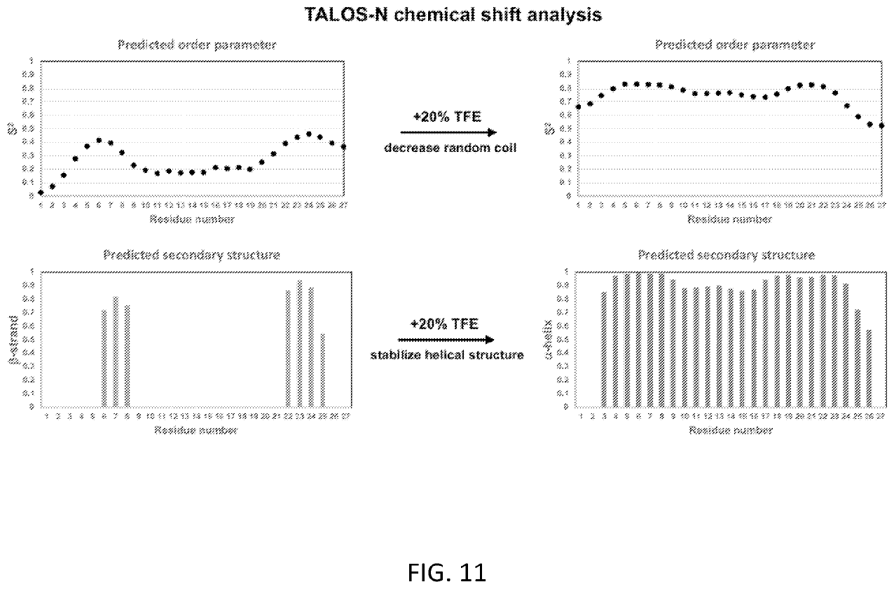

A full assignment of peptide structure was performed on peptide 17x-3 at 5 mg/ml and is shown in FIG. 10. Specific spectra, analysis and residue assignments are shown in FIG. 10, which include ¹⁵N-HSQC, ¹³C-HSQC, TOCSY/NOESY H_{N}-H_{α} regions, and NOESY H_{N} regions. A start comparison is noted with addition of 20% TFE as peaks shift drastically to ordered helical structures. These data were used in a TALOS-N chemical shift analysis, presented in FIG. 11, to predict the secondary shape amino acid residue. Two beta-sheet regions are present at the beginning (aa5-8) and end of the sequence (aa22-25), while highly ordered alpha-helices are noted across the entire peptide with the addition of TFE. The two beta-sheet regions correspond to uncoiled regions noted in our in silico simulation (FIG. 9c. bottom right peptide for example).

### Discussion

### Peptide Binding Intensity and Specificity

Originally, these peptides were designed with the intent of producing multiple HA, or B(X₇)B, binding domains, which are generally present alpha-helical secondary structures. Peptides 6f-2 (6 domains), 8h-2(7 domains), and 17x-3 (5 domains) all formed helical peptides in some form (FIG. 1). However, not all peptides bound comparably to mPEP35. In particular, 8h-2 and 17x-3 were proficient and specific binders, with 17x-3 being a significantly stronger binder at higher concentrations (5-10 mg/ml). Without wishing to be bound by theory, it is possible that (1) there may be an optimal number of B(X₇)B domains within comparable sequence lengths, and/or (2) full helical structure isn't necessary in HA binding and the non-helical N and C-termini branches might impact binding. With these concepts, other peptides were designed with a similar number of domains. Again, it was noted that peptides with comparable binding, especially 4 and BHP4, had similar features (free N and C-termini with a helical core) in the models presented. Furthermore, higher concentrations also promoted more potent binding and for each of these peptides, films were noted to have developed onto the HA coatings. It may be possible that some higher order structures are forming at these concentrations. It may also be forming a complex with HA. Therefore, secondary and higher order nanostructures were evaluated to investigate this relationship.

### Peptide Secondary Conformations Using Circular Dichroism

Increasing the concentration of a peptide can cause conformational switching from α-helices to β-sheet conformation. This conformational switching was observed for all the peptides that were tested, i.e., the CD profiles changed when going from 1.25 mg/ml to 2.5 mg/ml and then to 5 mg/ml, suggesting a change in conformation of the peptides based on the fact that the minimums change from below 210 nm at the low concentrations to above 220 nm in the higher concentrations.

It is possible that there are two characteristics for a 3₁₀ helix: (1) a [θ]_{222 nm}/[θ]₂₀₇ₙₘ ratio of 0.4, and (2) a shoulder near 222 nanometers. Furthermore, a [θ]_{222 nm}/[θ]_{208 nm} ratio is indicative of an α-helix in the range of 0.85-0.95. 3₁₀ helices are usually short sequences (typically 3 residues); although, segments as long as 11 residues may be possible. Molecular dynamics suggests that 3₁₀ helices and α-helices may coexist in the same peptide. As shown in Table 2, in the presence of 20 mM sodium cacodylate and 1.25 mg/ml, all the peptides demonstrate [θ]_{222 nm}/[θ]_{208 nm} ratio ratios that are between 0.42 to 0.81. This suggests that each of these peptides are apparently composed of individual molecules that are partially 3₁₀ helix and α-helix. Additionally, those peptides that have high ratios, e.g., BHP3 (0.79) and BHP4 (0.81), apparently contain more α-helix than 3₁₀helix.

Peptides in solution which are predominantly random coils can be induced to form either an α-helix or β-sheet conformation with the addition of TFE. Moreover, if a peptide shows secondary structure, addition of more TFE increases the ellipticities, i.e., become more negative, indicating more structure. As shown in Table 2, 20 mM SC, 20% TFE at 1.25 mg/ml, demonstrates ellipticity values that are considerably more negative than in 20 mM sodium cacodylate alone, suggesting more secondary structure. From that same data set, the ratios for most of the peptides range between 0.88 and 0.97, suggesting that the TFE has caused these peptides to form α-helices. There are three exceptions, i.e., 17x-3 (0.72), 6f-2 (0.84) and 10K (0.83), suggesting that TFE has not caused these peptides to become fully helical.

Beta-sheets typically have a CD minimum between 217 to 220 nanometers and typically a band at 218; although, β-sheets can demonstrate variable CD profiles which are in part due to the type of β-sheets (anti-parallel or parallel) and the degree of twist of the conformation of the peptide. The CD minimums for all the peptides at 5.0 mg/ml ranged from 221 to 226 nanometers (with one exception, i.e., peptide 6b is 227 nanometers). These profiles suggest that the peptides in 20 mM sodium cacodylate or 20 mM sodium cacodylate/20% TFE at 5.0 mg/ml are conformationally twisted β-sheets.

### Solution-Bound Hyaluronic Acid Loss Using Nuclear Magnetic Resonance

Although the binding results showed a clear difference in peptide affinity for HA, solution-based peptide binding complexes are more likely to be a natural event. When comparing the most significant peptide binder 17x-3 to the controls, the HA was almost entirely removed from solution at concentrations 2.5 to 5 mg/mL peptide as was most of the peptide (˜90%). The positive control mPEP35 could only pull at most 40% of the peptide in any condition while the negative control 1-scrm was, unexpectedly, able to remove the HA with 5 mg/mL peptide. This reflects the significant binding result noted for 17x-3 at 5 mg/mL (FIG. 3) and the shift to beta sheet at 2.5 and 5 mg/mL (FIG. 4), which suggests that the structure may play a component in both the binding and the forming of an insoluble HA complex. The fact that 1-scrm does not bind but is a able to pull HA out of solution at 5 mg/mL could be an event caused by positively charged complexes noted in other peptides, polypeptides, or even polyeletrolyte complexes formed with chitosan.

### Higher Order Nanostructures Using Transmission Electron Microscopy

There are four peptides that predominantly form fibers with striations and furthermore form a mesh network, specifically 17x-3, 4, BHP3 and BHP4. The hydrophobic residues may be exposed in these conditions, noted in the helical net figures, thus promoting higher order nanofiber formation. Given that TEM demonstrates a mesh network and the binding data suggest that a layer of peptide is formed in the HA binding assay, it is possible that HA can cause a non-covalent crosslinking of the peptide to form a self-supporting-hydrogel. Since HA is predominant in brain tissue, this scaffold could be used for central nervous system derived cell encapsulation and neural tissue engineering applications.

### Corroboration of Secondary and Higher Order Structure Using FTIR

The CD data indicates that there is an apparent concentration dependant shift in conformation from a 3₁₀-helix at 1.25 mg/mL to a β-sheet at 5.0 mg/mL. Two distinct signatures at ≈1695-1675 cm⁻¹ and ≈1638-1632 cm⁻¹ or 1640-1610 cm⁻¹ signify an antiparallel β-sheet. More specifically, i.e., 1685, 1641 and 1636 cm⁻¹[55] for intramolecular β-sheets and (ii) 1695 and 1625, 1625-1615 cm⁻¹[56] or 1692 and 1625 cm⁻¹ for intermolecular β-sheets. In general terms, the wave numbers at ≈1640-1630 cm⁻¹ demonstrate a strong signal (S) and those at ≈1695 or ≈1685 demonstrate a weak signal (W). Examination of the wave number data in Table 4 indicates that for all of the peptides analysed, there is both a strong (S) and weak (W) FTIR signal, suggesting that the β-sheet formed by these peptides are antiparallel. β-sheets can have a twist which is primarily due to intrastrand non-bonded interactions which causes the β-sheet to adopt a right handed twist, specifically inter-atomic interactions involving the C^{β}H₃, groups of amino acids like Ala or C^{γ}H₃ as in the amino acids Leu and Ile. The peptides in this study contain the amino acids Ala, Leu and Ile, and the CD data suggests that at 5.0 mg/mL, the peptides form twisted antiparallel β-sheets (i.e., the CD demonstrates a band at greater than 220 nm). In addition, in SC containing TFE, the fibers formed are clearly twisted structures (much like a twisted ribbon. Furthermore, the peptides exhibited higher ordered structures (1690 and 1640-1610 cm⁻¹ [52]) such as aggregated strands (1695-1675 cm⁻¹) and the presence of fibers at 3300-3270 cm⁻¹ or 1625-1614 cm⁻¹ for all of the peptides reported in Table 4.

A partially folded helical intermediate may be responsible for the transformation of Aβ fibrils, specifically: Aβ monomers→aggregates→fibrils. Peptides that are in the process of folding into a secondary structure may contain random coils and turns with a progressive transition to 3₁₀-helices and finally to the more stable α-helix, i.e., a so called 3₁₀ helix→α-helix switching event. 3₁₀ helices may be a transient intermediate in the formation of a 3₁₀-to-β-sheet conformational transition. The FTIR data for the peptides in this study indicate that all of these structural elements are present, suggesting a transition pathway to the formation of the self-assembled peptides observed in the TEM.

### Modelling and NMR Predictions

The data presented herein indicate that the dominant monomeric secondary structure is helical (both α and 3₁₀) and beta sheets (anti-parallel) are likely more prevalent in the higher order self-assembled nanofibers. It should be noted that the helical probabilities are high for nearly every region (>0.85) of the proposed B(X₇)B domains (FIG. 2C), which suggests that all of the domains are binding candidates.

### SUMMARY

In this work, a class of peptides was designed with helical presenting B(X₇)B hyaluronic acid binding domains, to create robust hyaluronic acid (HA) binding behaviour. Ten different peptides were assessed. Simple molecular modelling was used to evaluate secondary structures, concentration and extra-cellular dependent binding assay were performed, concentration mediated secondary structures were assessed using circular dichroism, fourier transform intrared, and nuclear magnetic resonance spectroscopy (CD/FTIR/NMR), and higher order nanostructures were visualized using transmission electron microscopy (TEM). All peptides formed the initial apparent 3₁₀/helical shapes, however peptides termed 17x-3, 4, BHP3 and BHP4 were found to be HA specific and robust binders, especially at higher concentrations. It was noted from CD-derived ellipticity ratios, that these peptides shifted from the initially apparent 3₁₀/alpha-helical shapes at lower concentrations (1.25 mg/mL) to anti-parallel beta-sheets at higher concentrations (5 mg/mL). It was also at these beta-sheet forming concentrations when 17x-3 mixed in solution with HA, was able to form a complex and pull all HA out of solution. Furthermore, these binders also formed nanofibers commonly noted as self-assembling structures typically cued by beta-sheet formation. As such, HA binding peptides are provided herein that outperform mPEP35 (positive commercial standard) 3-4 times at higher concentrations, which was enhanced by self-assembly.

### Tables

TABLE 1
Design of Peptides

| | | HA binding | | |
|---|---|---|---|---|
| | | domains [B(X₇)B]: | Number | Hydrophobicity |
| | | contiguous; non- | of | to |
| Peptide | Amino acid Sequence | contiguous | Residues | Hydrophilicity |
| mPEP35¹ | LKQKIKHVVKLKVVVKLRSQLVKRKQN | (i) K2-K10, (ii) K10- | 27 | 1:1.3 |
| | (SEQ ID NO: 6) | R18; (iii) K4-K12, | | |
| | | (iv) K16-R24 | | |
| 1-scrm² | KKKKKLQLQLNLIKKKVQVSVVVVRRH | (i) No HA, B(X₇)B, | 27 | 1:1.3 |
| | (SEQ ID NO: 7) | binding domains | | |
| 17x-3 | KTKATVLIKNKQKSKNALKQKIVLLSK | (i) K3-K11, (ii) K11- | 27 | 1:1.3 |
| | (SEQ ID NO: 1) | K19, (iii) K19-K27; | | |
| | | (iv) K1-K9, (v) K13- | | |
| | | K21 | | |
| 6f-2 | TKSKIKIVVKSKAKKLRLALVKRIKI | (i) K6-K14, (ii) K14- | 26 | 1:1 |
| | (SEQ ID NO: 8) | K22; (iii) K2-K10, | | |
| | | (iv) K4-K12, (v) | | |
| | | K15-R23, | | |
| | | (vi) R17-K25 | | |
| 8h-2 | TKTKIKIIVKLKSKAKLRIKLVKRHKS | (i) K2-K10, (ii) K10- | 27 | 1:1.1 |
| | (SEQ ID NO: 9) | R18, (iii) R18-K26 & | | |
| | | (iv) K4-K12, (v) | | |
| | | K12-K20; (vi) K6- | | |
| | | K14, (vii) K16-R24 | | |
| 2^{nd} de | ASNLTKAAKSLKVRVIKKTKQKQVLKVL | (i) K6-R14, (ii) R14- | 28 | 1:1 |
| novo | (SEQ ID NO: 10) | K22; (iii) K9-K17, | | |
| | | (iv) K12-K20, (v) | | |
| | | K18-K26 | | |
| 10K | TKAKIKHSVKLKAKLRIKLVKRI | (i) K2-K10, (ii) K10- | 23 | 1:0.9 |
| | (SEQ ID NO: 11) | K18 & (iii) K6-K14, | | |
| | | (iv) K14-R22; (v) | | |
| | | K4-K12 | | |
| 4³ | LKTKIKIIVKTKSSAKLRSKLVNSHKI | (i) K2-K10, (ii) K10- | | |
| | (SEQ ID NO: 2) | R18, (iii) R18-K26 & | 27 | 1:1.1 |
| | | (iv) K4-K12, (v) | | |
| | | K12-K20 | | |
| 6b⁴ | LKLKSSHSIKLKVKSKQRSALVSRQKA | (i) K2-K10, (ii) K10- | 27 | 1:1.7 |
| | (SEQ ID NO: 12) | R18, (iii) R18-K26; | | |
| | | (iv) K4-K12 & (v) | | |
| | | K16-R24 | | |
| 7c⁵ | KTKATVKIKNKQKSVNALKQKIVLLSK | (i) K3-K11, (ii) K11- | 27 | 1:1.3 |
| | (SEQ ID NO: 5) | K19, (iii) K19-K27; | | |
| | | (iv) K1-K9, (v) K13- | | |
| | | K21 | | |
| BHP3⁶ | TQLRNKYTFLARARNALAVRTKQNIKS | (i) R4-R12, (ii) R12- | 27 | 1:0.9 |
| | (SEQ ID NO: 3) | R20 and (iii) K6-14R, | | |
| | | (iv) R14-K22 | | |
| BHP4⁷ | TNLRNKYTFLARARANLAVRNKQNIKS | (i) R4-R12, (ii) R12- | 27 | 1:1.1 |
| | (SEQ ID NO: 4) | R20 and (iii) K6-14R, | | |

¹mPEP35 is the reference peptide and is the same sequence as from Zaleski et al.
²1-scrm is a scrambled version of mPEP35, so that there are no HA binding domains and is the same sequence as from Lee et al.¹³
³Peptide 4 is an 8h-2 variant.
⁴Peptide 6b is an mPEP35 variant.
⁵Peptide 7c is a 17x-3 variant.
⁶Peptide BHP3 is designed from a 42 residue peptide BH-Pas from Xu et al.¹⁴
⁷Peptide BHP4 is a variant of BHP3.

TABLE 2
Ellipticity ratio(222 nm/208 nm) at 1.25 mg/mL and
wavelength minimum for 5.0 mg/mL from CD Profiles

| | Peptide in 20 mM Sodium Cacodylate | | | | Peptide in 20 mM Sodium Cacodylate, 20% TFE | | | |
|---|---|---|---|---|---|---|---|---|
| | 1.25 mg/mL | | | 5.0 mg/mL | 1.25 mg/mL | | | 5.0 mg/mL |
| Peptide | 222 nm | 208 nm | Ratio¹ | Minimum² | 222 nm | 208 nm | Ratio¹ | Minimum² |
| mPEP35 | −51.9 | −105 | 0.49 | 225 | −214 | −232 | 0.92 | 223 |
| 1-scrm | −63.2 | −117 | 0.54 | 225 | −249 | −277 | 0.90 | 223 |
| 17x-3 | −64.4 | −135 | 0.48 | 223 | −126 | −175 | 0.72 | 221 |
| 6f-2 | −50.6 | −97.6 | 0.52 | 221 | −257 | −306 | 0.84 | 221 |
| 8h-2 | −57.3 | −135 | 0.42 | 224 | −249 | −283 | 0.88 | 222-224 |
| 2^{nd} DN | −48.5 | −115 | 0.42 | 224-225 | −333 | −371 | 0.90 | 221-222 |
| 10K | −75.3 | −113 | 0.67 | 224 | −194 | −235 | 0.83 | 223 |
| 4 | −60.3 | −112 | 0.54 | 225 | −353 | −364 | 0.97 | 224 |
| 6b | N/A ³ | N/A ³ | N/A ³ | 227 | −199 | −229 | 0.87 | 224 |
| 7c | −60.9 | −131 | 0.46 | 225 | −259 | −290 | 0.89 | 224 |
| BHP3 | −70.1 | −88.4 | 0.79 | 226 | N/A ³ | N/A ³ | N/A ³ | 226 |
| BHP4 | −86.6 | −107 | 0.81 | 226 | N/A ³ | N/A ³ | N/A ³ | 225 |

¹Ellipticity Ratio: The ratio of molar ellipticities at 222 nm and 208 nm is done as an indication of the potential secondary structure of the peptide, i.e., a ratio of 0.85-0.95 is typical ofα-helices.²⁰ As well, a 222/207 ratio of 0.4 is indicative of a 3₁₀ helix, as well as a shoulder at approximately 222 nm.²¹ Toniolo also indicates a blue shift of 1 nanometer for the band near 208 nm. Six of the peptides show this blue shift in wavelength (to the nearest nm), specifically: 207 nm for mPEP35, 1-scrm, 17x-3, 6f-2, 8h-2, 2^{nd} denovo, 7c; others do not, 208 nm for peptide 4. The remainder are as follows: 10K (209 nm), BHP3 (209 nm) and BHP4 (210 nm), although, their ratio is well above the level of the other peptides suggesting that the CD profiles contain more α-helix than 3₁₀ helix.
²The wavelength of the minimum for the 5.0 mg/mL CD profile is reported in nanometers (nm).
³ The wavelength minimums for peptide 6b at 1.25 mg/mL are considerably displaced from the typical 222 nm and 208 nm, specifically 227 nm and 206 respectively; thus, the 222 nm/208 nm ratio was not calculated for this concentration of the peptide. The wavelength minimums for peptide BHP3 and BHP4 at 1.25 mg/mL suggest that a secondary structural change has occurred so that calculating a ratio for α-helices is not valid for this concentration of the peptide.

TABLE 3
Transmission electron microscopy (TEM) characterization

| | peptide | Phosphate Buffered Saline (i) | Sodium Cacodylate Buffer (ii) |
|---|---|---|---|
| A | mPEP35 | Structure containing a small number of | No structure or fibrils |
| | | striations (2 fibrils mostly, up to 5 in | |
| | | some cases) that go to form larger fibers | |
| B | 1-scrm | Two stranded fibril, that perhaps forms | No structure or fibrils |
| | | fibers of multiple fibrils | |
| C | 17x-3 | Fibers with many striations, that form | Fibers that look like a twisted ribbon |
| | | larger fibers. The larger fibers tend to | |
| | | form a mesh network | |
| D | 6f-2 | Structure, but no discernible striations | No structure or fibrils |
| E | 8h-2 | No discernible structure. | No discernible structure. |
| F | 2^{nd} DN | Structure, but no discernible striations | No structure |
| G | 10K | No structure | Some very thin fibrils |
| H | 4 | Fibers containing two striations. The | Three twisted fibers, right next to each other |
| | | two stranded fibers form a mesh | |
| | | network or can form a mesh network of | |
| | | larger sized fibers | |
| I | 6b | Structure with striations | Two strands of very fine fibrils, that is twisted |
| | | | into a fiber |
| J | 7c | Fibrils with striations that form larger | Fibers that look like a twisted ribbon |
| | | fibers | |
| K | BHP3 | Fibers with many striations that form | Fibers that look like twisted ribbons |
| | | larger fibers. The larger fibers tend to | |
| | | form a mesh network | |
| L | BHP4 | Fibers with many striations that form | Fibers that look like twisted ribbons |
| | | larger fibers. The larger fibers tend to | |
| | | form a mesh network | |

TABLE 4
Fourier Transform Infrared (FTIR) Peak Analysis for Peptides in 20% TFE

| | Conc. | | | β-sheet, antiparallel⁴ | | [symbol] /aggreg | |
|---|---|---|---|---|---|---|---|
| Peptide¹ | (mg/ml) | α-helix ² | 3₁₀ helix ³ | weak (W) | strong (S) | sh/fib; a/strand⁵ | Fiber⁶ |
| mPEP35 | 1.25 | 1649², 1670⁷ | 1661, 1670⁷ | 1686 | 1630 | 1612 | 3296^{b} |
| | 2.5 | 1649², 1671⁷ | 3500^{c}, 1661, | 1682, [symbol] | 1633, 1637 | [symbol] | 3285^{b}, 1622 |
| | | | 1671⁷ | | | | |
| | 5 | 1650², 1671⁷ | 1662, 1671⁷ | [symbol] 1681 | 1631, 1640 | [symbol] | 3288^{b}, 1621 |
| 1scrm | 1.25 | 1648², 1670⁷ | 3500^{c}, 1661, | [symbol] 1681 | 1630, 1640 | [symbol] | 3277^{b}, 1621, |
| | | | 1670⁷ | | | | 1693 |
| | 2.5 | 1640², 1649², | 1660, 1670⁷ | 1685 | 1632, 1640 | 1617 | 3276^{b}, 1617 |
| | | 1670⁷ | | | | | |
| | 5 | 1649², 1660², | 1660, 1670⁷ | 1681, [symbol] | 1630, 1639 | [symbol] | 1620 |
| | | 1670⁷ | | | | | |
| 17x3 | 1.25 | 1650², 1671⁷ | 1662, 1671⁷ | 1681, [symbol] | 1631, 1639 | 1610, [symbol] | 3288^{b}, 1621 |
| | 2.5 | 1649², 1671⁷ | 3500^{c}, 1661, | 1694, 1682 | 1630, 1639 | [symbol] | 3276^{b}, 1621 |
| | | | 1671⁷ | | | | |
| | 5 | 1651², 1671⁷ | 1661, 1671⁷ | 1681, [symbol] | 1632 | 1610 [symbol] , | 3275^{b}, 1619 |
| | | | | | | [symbol] | |
| 4 | 1.25 | 1650², 1660², | 1660, 1671⁷ | 1681, [symbol] | 1629 | 1610, [symbol] | 3293^{b}, 1619 |
| | | 1671⁷ | | | | | |
| | 2.5 | 1649², 1660², | 1660, 1671⁷ | 1681, [symbol] | 1629, 1639 | [symbol] | 3293^{b} |
| | | 1671¹² | | | | | |
| | 5 | 1647², 1670⁷ | 1660, 1670⁷ | 1681, [symbol] | 1630 | [symbol] | 1620 |
| BHP3 | 1.25 | 1649², 1670⁷ | 1660, 1670⁷ | 1682, [symbol] | 1630, 1639 | [symbol] | 3286^{b}, 1620 |
| | 2.5 | 1649², 1671⁷ | 1661, 1671⁷ | 1681, [symbol] | 1631, 1637 | [symbol] | 3288^{b}, 1621 |
| | 5 | 1650², 1671⁷ | 1660, 1671⁷ | 1681, [symbol] | 1629, 1639 | [symbol] | 3279^{b}, 1619 |
| BPH4 | 1.25 | 1650², 1671⁷ | 1660, 1671⁷ | 1682, [symbol] | 1631 | 1610, [symbol] | 3284^{b}, 1621 |
| | 2.5 | 1649², 1671⁷ | 1660, 1671⁷ | 1530^{d}, 1681, | 1629, 1639 | [symbol] | 1619 |
| | | | | [symbol] | | | |
| | 5 | 1649², 1660², | 3500^{c}, 1660, | 1681, [symbol] | 1628, 1637 | 1610, [symbol] | 3279^{b} |
| | | 1670⁷ | 1670⁷ | | | | |

¹Peptide concentrations - 1.25, 2.50 and 5.0 mg/mL. Values reported are in wavenumbers (cm⁻¹), for convenience some cm⁻¹ have been reported as two values, e.g., 1661/71 means 1661 & 1671 cm⁻¹ [52].
² α-helix - Range of wavenumbers: (i) 1651 cm⁻¹ and 1650-1649 cm⁻¹ [57], (ii) 1660-1648 cm⁻¹.
³ 3₁₀ helix Range of wavenumbers: (i) 1665-1655 cm⁻¹ [36]; (ii) 1670-1660 cm⁻¹ [39].
⁴Weak (W) and strong (S) denotes an antiparallel β-sheet as defined by the two distinct wavenumbers which are underlined in the table as described by the following wavenumbers: (i) 1695-1675 cm⁻¹ (W, antiparallel) and 1638-1632 cm⁻¹(S, antiparallel) [36]; (ii) 2 distinct bands near 1690 cm⁻¹ and near 1630 cm⁻¹ define antiparallel β-sheets [58]; (iii) 2 distinct bands at ~1,690 cm⁻¹ and1,640-1,610 cm⁻¹ [52]. Intramolecular β-sheet: 1685 & 1641/1636 cm⁻¹ [55], 1689 & 1639 [56], 1686 & 1629 [59], 1686-1680 [60] and 1695-1675 & 1638-1632 [36].
⁵inter/aggreg sh/fib; a/strand - Intermolecular aggregated β-sheet (fiber), i.e., [inter/aggreg sh/fib] at 1627-1615 cm⁻¹ [36]; aggregated strands, i.e., [a/strand] at 1628-1610 cm⁻¹ [39]; 1692 & 1625 cm⁻¹ [55]; 1695& 1625, 1625-1615 cm⁻¹ [56]. Dashed underlines indicate intermolecular aggregated β-sheet.
⁶Fiber - defined at3300-3270 cm⁻¹, 1693 cm⁻¹ (W, antiparallel β-sheet) and 1625-1614 cm⁻¹ (S, antiparallel amide I hydrogen bonding) and 1537 cm⁻¹ (antiparallel amide II) [51].
⁷Denotes a combination of an α-helix/3₁₀ helix conformation at 1673-1670 cm⁻¹ [49, 53, 54].
^{a} α-helix, 3502 cm⁻¹, NH stretch [51].
^{b}Fiber, hydrogen bonded amides, 3300-3270 cm⁻¹ [51].
^{c}3₁₀ helix, 3500 cm⁻¹, NH stretch [51].
^{d}Beta sheet 1530 cm⁻¹ [61]

TABLE 5
Peptide Concentration Calculations

| | | | | | | mass: mg/mmole | |
|---|---|---|---|---|---|---|---|
| | | | | | | peptide, Biotin N-ter, | |
| | | | | | | free C-ter | |
| | | Conc. | Peptide Masses (mg/mmole) | | | combined −H2O(Nterm) + | Conc |
| # | | (mg/ml) | mass | biotin | combined | H2O (Cterm) | (mMolar) |
| 1 | mPEP35 | 1.25 | 3221.1 | 244.31 | 3465.41 | 3465.41 | 0.36 |
| | | 2.5 | 3221.1 | 244.31 | 3465.41 | | 0.72 |
| | | 5 | 3221.1 | 244.31 | 3465.41 | | 1.44 |
| 2 | 1-scrm | 1.25 | 3221.1 | 244.31 | 3465.41 | 3465.41 | 0.36 |
| | | 2.5 | 3221.1 | 244.31 | 3465.41 | | 0.72 |
| | | 5 | 3221.1 | 244.31 | 3465.41 | | 1.44 |
| 3 | 17x-3 | 1.25 | 3051.8 | 244.31 | 3296.11 | 3296.11 | 0.38 |
| | | 2.5 | 3051.8 | 244.31 | 3296.11 | | 0.76 |
| | | 5 | 3051.8 | 244.31 | 3296.11 | | 1.52 |
| 4 | 6f-2 | 1.25 | 2990 | 244.31 | 3234.31 | 3234.31 | 0.39 |
| | | 2.5 | 2990 | 244.31 | 3234.31 | | 0.77 |
| | | 5 | 2990 | 244.31 | 3234.31 | | 1.55 |
| 5 | 8h-2 | 1.25 | 3187.1 | 244.31 | 3431.41 | 3431.41 | 0.36 |
| | | 2.5 | 3187.1 | 244.31 | 3431.41 | | 0.73 |
| | | 5 | 3187.1 | 244.31 | 3431.41 | | 1.46 |
| 6 | 2^{nd} deno | 1.25 | 3121.9 | 244.31 | 3366.21 | 3366.21 | 0.37 |
| | | 2.5 | 3121.9 | 244.31 | 3366.21 | | 0.74 |
| | | 5 | 3121.9 | 244.31 | 3366.21 | | 1.49 |
| 7 | 10K | 1.25 | 2700.5 | 244.31 | 2944.81 | 2944.81 | 0.42 |
| | | 2.5 | 2700.5 | 244.31 | 2944.81 | | 0.85 |
| | | 5 | 2700.5 | 244.31 | 2944.81 | | 1.70 |
| 8 | 4 | 1.25 | 3062.8 | 244.31 | 3307.11 | 3307.11 | 0.38 |
| | | 2.5 | 3062.8 | 244.31 | 3307.11 | | 0.76 |
| | | 5 | 3062.8 | 244.31 | 3307.11 | | 1.51 |
| 9 | 6b | 1.25 | 3049.7 | 244.31 | 3294.01 | 3294.01 | 0.38 |
| | | 2.5 | 3049.7 | 244.31 | 3294.01 | | 0.76 |
| | | 5 | 3049.7 | 244.31 | 3294.01 | | 1.52 |
| 10 | 7c | 1.25 | 3037.8 | 244.31 | 3282.11 | 3282.11 | 0.38 |
| | | 2.5 | 3037.8 | 244.31 | 3282.11 | | 0.76 |
| | | 5 | 3037.8 | 244.31 | 3282.11 | | 1.52 |
| 11 | BHP3 | 1.25 | 3162.7 | 244.31 | 3407.01 | 3407.01 | 0.37 |
| | | 2.5 | 3162.7 | 244.31 | 3407.01 | | 0.73 |
| | | 5 | 3162.7 | 244.31 | 3407.01 | | 1.47 |
| 12 | BHP4 | 1.25 | 3161.7 | 244.31 | 3406.01 | 3406.01 | 0.37 |
| | | 2.5 | 3161.7 | 244.31 | 3406.01 | | 0.73 |
| | | 5 | 3161.7 | 244.31 | 3406.01 | | 1.47 |

TABLE 6
Fourier Transform Infrared (FTIR) Peak Analysis for Peptides without 20% TFE

| | Conc. | | | β-sheet, antiparallel⁴ | | [symbol] /aggreg | |
|---|---|---|---|---|---|---|---|
| Peptide¹ | (mg/ml) | α-helix ² | 3₁₀ helix ³ | weak (W) | strong (S) | sh/fib; a/strand⁵ | Fiber ⁶ |
| mPEP35 | 1.25 | 3502^{a}, 1648², | 1661, 1671⁷ | 1682, [symbol] | 1629 | [symbol] | 1620 |
| | | 1671⁷ | | | 1637, 1640 | | |
| | 2.5 | 1650², 1671⁷ | 1660 | 1681, [symbol] | 1630 | [symbol] | 3273^{b} |
| | 5 | 1650², 1671⁷ | 3500^{c}, 1661 | 1530^{d}, 1682, | 1631, 1640 | [symbol] | 1621 |
| | | | | [symbol] | | | |
| 1scrm | 1.25 | 3502^{a}, 1659² | 1659, 1671⁷ | 1681, [symbol] | 1632 | 1618 | 3276^{b}, 1618 |
| | | 1671⁷ | | | | | |
| | 2.5 | 1659², 1671⁷ | 1659, 1671⁷ | 1682, [symbol] | 1632 | [symbol] | 3278^{b}, 1617 |
| | 5 | 1659², 1670⁷ | 1659, 1670⁷ | 1682, [symbol] | 1632 | [symbol] | 3278^{b}, 1617 |
| 17x3 | 1.25 | 1650, 1671⁷ | 1661, 1671⁷ | 1682, [symbol] | 1631, 1640 | [symbol] | 3280^{b}, 1620 |
| | 2.5 | 1650², 1671⁷ | 3500^{c}, 1671⁷ | 1530^{d}, 1682, | 1631 | 1610, [symbol] | 3277^{b}, 1621 |
| | | | | [symbol] | 1639 | | |
| | 5 | 1650², 1671⁷ | 1661, 1671⁷ | 1682, [symbol] | 1630, 1640 | [symbol] | 3273^{b}, 1619 |
| 4 | 1.25 | 1650², 1671⁷ | 1661, 1671⁷ | 1681, [symbol] | 1630 | [symbol] | 3275⁶, 1620 |
| | | | | | 1640 | | |
| | 2.5 | 1651², 1671⁷ | 1661, 1671⁷ | 1682, [symbol] | 1630 | [symbol] | 3283^{b}, 1620 |
| | 5 | 1650², 1671⁷ | 1661, 1671⁷ | 1681, [symbol] | 1631 | 1610, [symbol] | 1621 |
| | | | | | 1639 | | |
| BHP3 | 1.25 | 1659², 1671⁷ | 1659, 1671⁷ | 1681, [symbol] | 1632 | [symbol] | 3275^{b}, 1616 |
| | 2.5 | 1659², 1670⁷ | 1659, 1670⁷ | 1682, [symbol] | 1632 | [symbol] | 3274^{b}, 1617 |
| | 5 | 1659², 1671⁷ | 1659, 1671⁷ | 1682, [symbol] | 1632 | [symbol] | 1617 |
| BPH4 | 1.25 | 1659², 1671⁷ | 1659, 1671⁷ | 1681, [symbol] | 1632 | [symbol] | 1617 |
| | 2.5 | 1659², 1671⁷ | 1659, 1671⁷ | 1681, [symbol] | 1632 | [symbol] | 3273^{b}, 1617 |
| | 5 | 1659², 1670⁷ | 1659, 1670⁷ | 1681, [symbol] | 1632 | [symbol] | 3273^{b}, 1617 |

¹Peptide concentrations - 1.25, 2.50 and 5.0 mg/mL. Values reported are in wavenumbers (cm⁻¹), for convenience some cm⁻¹ have been reported as two values, e.g., 1661/71 means 1661 & 1671 cm⁻¹.
² α-helix - Range of wavenumbers: (i) 1651 cm⁻¹ and 1650-1649 cm⁻¹[57] , (ii) 1660-1648 cm⁻¹ [52].
³ 3₁₀ helix Range of wavenumbers: (i) 1665-1655 cm⁻¹ [34]; (ii) 1670-1660 cm⁻¹ [37].
⁴Weak (W) and strong (S) denotes an antiparallel β-sheet as defined by the two distinct wavenumbers which are underlined in the table as described by the following wavenumbers: (i) 1695-1675 cm⁻¹ (W, antiparallel) and 1638-1632 cm⁻¹(S, antiparallel) [34]; (ii) 2 distinct bands near 1690 cm⁻¹ and near 1630 cm⁻¹ define antiparallel β-sheets [58]; (iii) 2 distinct bands at ~1,690 cm⁻¹ and 1,640-1,610 cm⁻¹ [52]. Intramolecular β-sheet: 1685 & 1641/1636 cm⁻¹ [55], 1689 & 1639 [56], 1686 & 1629 [59], 1686-1680 [60] and 1695-1675 & 1638-1632 [34].
⁵inter/aggreg sh/fib; a/strand - Intermolecular aggregated β-sheet (fiber), i.e., [inter/aggreg sh/fib] at 1627-1615 cm⁻¹ [34]; aggregated strands, i.e., [a/strand] at 1628-1610 cm⁻¹ [37]; 1692 & 1625 cm⁻¹ [55]; 1695& 1625, 1625-1615 cm⁻¹ [56]. Dashed underlines indicate intermolecular aggregated β-sheet.
⁶ Fiber - defined at3300-3270 cm⁻¹, 1693 cm⁻¹ (W, antiparallel β-sheet) and 1625-1614 cm⁻¹ (S, antiparallel amide I hydrogen bonding) and 1537 cm⁻¹ (antiparallel amide II) [51].
⁷Denotes a combination of an α-helix/3₁₀ helix conformation at 1673-1670 cm⁻¹ [49, 53, 54].
^{a}α-helix, 3502 cm⁻¹, NH stretch [51].
^{b}Fiber, hydrogen bonded amides, 3300-3270 cm⁻¹ [51].
^{c}3₁₀ helix, 3500 cm⁻¹, NH stretch [51].
^{d}Beta sheet 1530 cm⁻¹ [61].

We claim:

1. A self-assembling peptide comprising a plurality of BX₇B domains, wherein B is a basic amino acid and X is any amino acid except an acidic amino acid, and wherein the self-assembling peptide binds to one or more extracellular matrix components.

2. The self-assembling peptide of claim 1, wherein the one or more extracellular matrix components are selected from collagen, elastin, and hyaluronic acid.

3. The self-assembling peptide of claim 2, wherein the self-assembling peptide binds to hyaluronic acid.

4. The self-assembling peptide of any one of the preceding claims, wherein the peptide comprises 26-28 amino acids.

5. The self-assembling peptide of any one of the preceding claims, comprising three, four or five BX₇B domains.

6. The self-assembling peptide of claim 5, comprising:
a) at least two contiguous BX₇B domains and at least two non-contiguous BX₇B domains;
b) a first set of at least two contiguous BX₇B domains and a second set of at least two contiguous BX₇B domains, each set is non-contiguous with the other set; or
c) at least three BX₇B domains, wherein two of the at least three BX₇B domains are contiguous with each other and the third BX₇B domain is non-contiguous with the other two BX₇B domains.

7. The self-assembling peptide of any one of the preceding claims, wherein B is a basic amino acid selected from histidine, arginine, or lysine.

8. The self-assembling peptide of any one of the preceding claims, wherein X is an amino acid selected from histidine, lysine, arginine, serine, threonine, asparagine, glutamine, alanine, valine, leucine, isoleucine, methionine, phenylalanine, tyrosine, tryptophan, proline, glycine, and cysteine.

9. The self-assembling peptide of any one of the preceding claims, wherein the peptide has a propensity to form β-sheet secondary structure.

10. The self-assembling peptide of any one of the preceding claims, comprising a sequence selected from:
(SEQ ID NO: 1)
KTKATVLIKNKQKSKNALKQKIVLLSK,
(SEQ ID NO: 2)
LKTKIKIIVKTKSSAKLRSKLVNSHKI,
(SEQ ID NO: 3)
TQLRNKYTFLARARNALAVRTKQNIKS,
(SEQ ID NO: 4)
TNLRNKYTFLARARANLAVRNKQNIKS,
and
(SEQ ID NO: 5)
KTKATVKIKNKQKSVNALKQKIVLLSK.

11. A nanofiber comprising a plurality of self-assembling peptides, wherein each of the plurality of self-assembling peptides comprises a plurality of BX₇B domains, wherein B is a basic amino acid and X is any amino acid except an acidic amino acid, wherein the self-assembling peptide binds to hyaluronic acid.

12. The nanofiber of claim 11, wherein each of the plurality of self-assembling peptides comprises three, four or five BX₇B domains.

13. The nanofiber of claim 11 or claim 12, wherein each of the plurality of self-assembling peptides comprises:
a) at least two contiguous BX₇B domains and at least two non-contiguous BX₇B domains;
b) a first set of at least two contiguous BX₇B domains and a second set of at least two contiguous BX₇B domains, each set is non-contiguous with the other set; or
c) at least three BX₇B domains, wherein two of the at least three BX₇B domains are contiguous with each other and the third BX₇B domain is non-contiguous with the other two BX₇B domains.

14. The nanofiber of any one of claims 11-13, wherein B is a basic amino acid selected from histidine, arginine, or lysine.

15. The nanofiber of any one of claims 11-14, wherein X is an amino acid selected from histidine, lysine, arginine, serine, threonine, asparagine, glutamine, alanine, valine, leucine, isoleucine, methionine, phenylalanine, tyrosine, tryptophan, proline, glycine, and cysteine.

16. The nanofiber any one of claims 11-15, wherein each self-assembling peptide has a propensity to form β-sheet secondary structure.

17. The nanofiber of any one of claims 11-16, wherein each self-assembling peptide comprising a sequence independently selected from:
(SEQ ID NO: 1)
KTKATVLIKNKQKSKNALKQKIVLLSK,
(SEQ ID NO: 2)
LKTKIKIIVKTKSSAKLRSKLVNSHKI,
(SEQ ID NO: 3)
TQLRNKYTFLARARNALAVRTKQNIKS,
and
(SEQ ID NO: 4)
TNLRNKYTFLARARANLAVRNKQNIKS,
and
(SEQ ID NO: 5)
KTKATVKIKNKQKSVNALKQKIVLLSK.

18. A system comprising the self-assembling peptide of any one of claims 1-10 bound to hyaluronic acid.

19. The system of claim 18, wherein the system comprises a hydrogel.

20. A material coated with the nanofiber of any one of claims 11-17, or the system of claim 18 or claim 19.

21. The material of claim 20, wherein the material comprises a biomedical device.

22. The material of claim 21, wherein the biomedical device comprises a neural implantable device.

23. A method of lubricating a biomaterial, the method comprising contacting the biomaterial with the system of claim 18.

24. The method of claim 23, wherein the biomaterial is a joint, a tissue, or an organ.

25. A method of treating an inflammatory condition in a subject, the method comprising providing to the subject the self-assembling peptide of any one of claims 1-10, the nanofiber of any one of claims 11-17, or the system of claim 18 or 19.

26. The method of claim 25, wherein the inflammatory condition is lung inflammation or arthritis.

27. A method of treating cancer in a subject, the method comprising providing to the subject the self-assembling peptide of any one of claims 1-10, the nanofiber of any one of claims 11-17, or the system of claim 18 or 19.

28. A method treating a wound in a subject, the method comprising providing to the subject the self-assembling peptide of any one of claims 1-10, the nanofiber of any one of claims 11-17, or the system of claim 18 or 19.
